SEQUENCE LISTING (62 sequences)

SEQ ID NO: 1 - RNA, 20 nt, synthetic construct
  misc_feature 1..20: note = Synthetic: sgRNA-RW
  source 1..20: mol_type = other RNA
gacggagggcatgtcgggct

SEQ ID NO: 2 - RNA, 20 nt, synthetic construct
  misc_feature 1..20: note = Synthetic: sgRNA-WT
  source 1..20: mol_type = other RNA
gacggagggcatgtcgggcc

SEQ ID NO: 3 - RNA, 20 nt, synthetic construct
  misc_feature 1..20: note = Synthetic: sgRNA-TM
  source 1..20: mol_type = other RNA
cttggcaaacagcatcttcc

SEQ ID NO: 4 - DNA, 20 bp, synthetic construct
  misc_feature 1..20: note = Synthetic: ATAD3A- RW_F4
  source 1..20: mol_type = other DNA
cctgcagccactccctgctc

SEQ ID NO: 5 - DNA, 21 bp, synthetic construct
  misc_feature 1..21: note = Synthetic: ATAD3A- RW_R4
  source 1..21: mol_type = other DNA
ccctcaacagaagctcccgcg

SEQ ID NO: 6 - DNA, 53 bp, synthetic construct
  misc_feature 1..53: note = Synthetic: ATAD3A- RW-, Illumina_F
  source 1..53: mol_type = other DNA
acactctttccctacacgacgctcttccgatctcctgcagccactccctgctc

SEQ ID NO: 7 - DNA, 53 bp, synthetic construct
  misc_feature 1..53: note = Synthetic: ATAD3A- RW-, Illumina_R
  source 1..53: mol_type = other DNA
gactggagttcagacgtgtgctcttccgatctccctcaacagaagctcccgcg

SEQ ID NO: 8 - DNA, 23 bp, synthetic construct
  misc_feature 1..23: note = Synthetic: ATAD3B- RW_F1
  source 1..23: mol_type = other DNA
ccggccacagaaggaaaacggtg

SEQ ID NO: 9 - DNA, 21 bp, synthetic construct
  misc_feature 1..21: note = Synthetic: ATAD3B- RW_R3
  source 1..21: mol_type = other DNA
ccctcaacagaagctcccaca

SEQ ID NO: 10 - DNA, 56 bp, synthetic construct
  misc_feature 1..56: note = Synthetic: ATAD3B-RW-, Illumina_F
  source 1..56: mol_type = other DNA
acactctttccctacacgacgctcttccgatctccggccacagaaggaaaacggtg

SEQ ID NO: 11 - DNA, 53 bp, synthetic construct
  misc_feature 1..53: note = Synthetic: ATAD3B-RW-, Illumina_R
  source 1..53: mol_type = other DNA
gactggagttcagacgtgtgctcttccgatctccctcaacagaagctcccaca

SEQ ID NO: 12 - DNA, 20 bp, synthetic construct
  misc_feature 1..20: note = Synthetic: ATAD3A- TM_F2
  source 1..20: mol_type = other DNA
ttcccgaggagccgagtctg

SEQ ID NO: 13 - DNA, 20 bp, synthetic construct
  misc_feature 1..20: note = Synthetic: ATAD3A- TM_R1
  source 1..20: mol_type = other DNA
gctctgcccagcgtccctgc

SEQ ID NO: 14 - DNA, 53 bp, synthetic construct
  misc_feature 1..53: note = Synthetic: ATAD3A-TM-, Illumina_F
  source 1..53: mol_type = other DNA
acactctttccctacacgacgctcttccgatctttcccgaggagccgagtctg

SEQ ID NO: 15 - DNA, 52 bp, synthetic construct
  misc_feature 1..52: note = Synthetic: ATAD3A-TM-, Illumina_R
  source 1..52: mol_type = other DNA
gactggagttcagacgtgtgctcttccgatctgctctgcccagcgtccctgc

SEQ ID NO: 16 - DNA, 20 bp, synthetic construct
  misc_feature 1..20: note = Synthetic: ATAD3B- TM_F1
  source 1..20: mol_type = other DNA
ccctgtcaccgaggcttccg

SEQ ID NO: 17 - DNA, 22 bp, synthetic construct
  misc_feature 1..22: note = Synthetic: ATAD3B- TM_R2
  source 1..22: mol_type = other DNA
agaagagtgaggggagacagaa

SEQ ID NO: 18 - RNA, 25 nt, synthetic construct
  modified_base 25: mod_base = OTHER; note = thymine
  misc_feature 1..25: note = Synthetic: Scrambled
  source 1..25: mol_type = other RNA
cgttaatcgcgtataatacgcgtat

SEQ ID NO: 19 - RNA, 27 nt, synthetic construct
  misc_feature 1..27: note = Synthetic: Scrambled
  source 1..27: mol_type = other RNA
atacgcgtattatacgcgattaacgac

SEQ ID NO: 20 - RNA, 25 nt, synthetic construct
  modified_base 24: mod_base = OTHER; note = thymine
  misc_feature 1..25: note = Synthetic: siRAD51
  source 1..25: mol_type = other RNA
gtcacaaactgatctaaaatgttta

SEQ ID NO: 21 - RNA, 27 nt, synthetic construct
  misc_feature 1..27: note = Synthetic: siRAD51
  source 1..27: mol_type = other RNA
taaacattttagatcagtttgtgacta

SEQ ID NO: 22 - RNA, 25 nt, synthetic construct
  misc_feature 1..25: note = Synthetic: siBRCA1
  source 1..25: mol_type = other RNA
gtacgagatttagtcaacttgttga

SEQ ID NO: 23 - RNA, 27 nt, synthetic construct
  misc_feature 1..27: note = Synthetic: siBRCA1
  source 1..27: mol_type = other RNA
tcaacaagttgactaaatctcgtactt

SEQ ID NO: 24 - RNA, 25 nt, synthetic construct
  modified_base 24..25: mod_base = OTHER; note = thymine
  misc_feature 1..25: note = Synthetic: siBRCA2
  source 1..25: mol_type = other RNA
caagaagcatgtcatggtaatactt

SEQ ID NO: 25 - RNA, 27 nt, synthetic construct
  misc_feature 1..27: note = Synthetic: siBRCA2
  source 1..27: mol_type = other RNA
aagtattaccatgacatgcttcttgag

SEQ ID NO: 26 - RNA, 25 nt, synthetic construct
  misc_feature 1..25: note = Synthetic: siCtIP
  source 1..25: mol_type = other RNA
gagaatgttttagatgacataaaga

SEQ ID NO: 27 - RNA, 27 nt, synthetic construct
  misc_feature 1..27: note = Synthetic: siCtIP
  source 1..27: mol_type = other RNA
tctttatgtcatctaaaacattctcag

SEQ ID NO: 28 - DNA, 20 bp, synthetic construct
  misc_feature 1..20: note = Synthetic: RAD51-F
  source 1..20: mol_type = other DNA
agaccgagccctaaggagag

SEQ ID NO: 29 - DNA, 20 bp, synthetic construct
  misc_feature 1..20: note = Synthetic: RAD51-R
  source 1..20: mol_type = other DNA
cttctctactcgcttgcccc

SEQ ID NO: 30 - DNA, 25 bp, synthetic construct
  misc_feature 1..25: note = Synthetic: CtIP-F
  source 1..25: mol_type = other DNA
cagaataggactgagtacggtaaag

SEQ ID NO: 31 - DNA, 22 bp, synthetic construct
  misc_feature 1..22: note = Synthetic: CtIP-R
  source 1..22: mol_type = other DNA
ctgactgccatcctttgtatct

SEQ ID NO: 32 - DNA, 20 bp, synthetic construct
  misc_feature 1..20: note = Synthetic: BRCA1-F
  source 1..20: mol_type = other DNA
aagctgacagatggttcatt

SEQ ID NO: 33 - DNA, 20 bp, synthetic construct
  misc_feature 1..20: note = Synthetic: BRCA1-R
  source 1..20: mol_type = other DNA
acaggttccttgatcaactc

SEQ ID NO: 34 - DNA, 20 bp, synthetic construct
  misc_feature 1..20: note = Synthetic: BRCA2-F
  source 1..20: mol_type = other DNA
tgtggaagttgcgtattgta

SEQ ID NO: 35 - DNA, 20 bp, synthetic construct
  misc_feature 1..20: note = Synthetic: BRCA2-R
  source 1..20: mol_type = other DNA
taaatctgatgatggacgcc

SEQ ID NO: 36 - DNA, 20 bp, synthetic construct
  misc_feature 1..20: note = Synthetic: Fig. 1B, sgRNA-RW (Variant-specific sgRNA)
  source 1..20: mol_type = other DNA
gacggagggcatgtcgggct

SEQ ID NO: 37 - DNA, 46 bp, synthetic construct
  misc_feature 1..46: note = Synthetic: Fig. 1B
  source 1..46: mol_type = other DNA
gtcgctcggctgacggagggcatgtcgggctgggagatcgctcagc

SEQ ID NO: 38 - DNA, 46 bp, synthetic construct
  misc_feature 1..46: note = Synthetic: Fig. 1B
  source 1..46: mol_type = other DNA
gctgagcgatctcccagcccgacatgccctccgtcagccgagcgac

SEQ ID NO: 39 - DNA, 31 bp, synthetic construct
  misc_feature 1..31: note = Synthetic: Fig. 1C, 5A, 12A
  source 1..31: mol_type = other DNA
tgacggagggcatgtcgggccgggagatcgc

SEQ ID NO: 40 - DNA, 37 bp, synthetic construct
  misc_feature 1..37: note = Synthetic: Fig. 3B
  source 1..37: mol_type = other DNA
tgacggagggcatgtcgggccgggagatcgctcagct

SEQ ID NO: 41 - DNA, 37 bp, synthetic construct
  misc_feature 1..37: note = Synthetic: Fig. 3B
  source 1..37: mol_type = other DNA
tgacggagggcatgtcggggctggaaaaccnttcact

SEQ ID NO: 42 - RNA, 20 nt, synthetic construct
  misc_feature 1..20: note = Synthetic: Fig. 6A sgRNA-TM (Variant-specific sgRNA)
  source 1..20: mol_type = other RNA
cttggcaaacagcatcttcc

SEQ ID NO: 43 - DNA, 46 bp, synthetic construct
  misc_feature 1..46: note = Synthetic: Fig. 6A
  source 1..46: mol_type = other DNA
cgggccaccaggcaccgggaagatgctgtttgccaaggtgagagcg

SEQ ID NO: 44 - DNA, 46 bp, synthetic construct
  misc_feature 1..46: note = Synthetic: Fig. 6A
  source 1..46: mol_type = other DNA
cgctctcaccttggcaaacagcatcttcccggtgcctggtggcccg

SEQ ID NO: 45 - DNA, 30 bp, synthetic construct
  misc_feature 1..30: note = Synthetic: Fig. 6B
  source 1..30: mol_type = other DNA
gcaccgggaagatgctgtttgccaaggtga

SEQ ID NO: 46 - DNA, 30 bp, synthetic construct
  misc_feature 1..30: note = Synthetic: Fig. 6B
  source 1..30: mol_type = other DNA
gcaccgggaagacgctgtttgccaaggtga

SEQ ID NO: 47 - RNA, 20 nt, synthetic construct
  misc_feature 1..20: note = Synthetic: Fig. 7A
  source 1..20: mol_type = other RNA
gacggagggcatgtcgggcc

SEQ ID NO: 48 - DNA, 45 bp, synthetic construct
  misc_feature 1..45: note = Synthetic: Fig. 7A ATAD3A WT / ATAD3B WT
  source 1..45: mol_type = other DNA
aggtcgctcggctgacggagggcatgtcgggccgggagatcgctc

SEQ ID NO: 49 - DNA, 45 bp, synthetic construct
  misc_feature 1..45: note = Synthetic: Fig. 7A ATAD3A c.1582C>T
  source 1..45: mol_type = other DNA
aggtcgctcggctgacggagggcatgtcgggctgggagatcgctc

SEQ ID NO: 50 - DNA, 300 bp, synthetic construct
  misc_feature 1..300: note = Synthetic: Fig. 11 ATAD3A
  source 1..300: mol_type = other DNA
tgcctgtcttccggcctccacctcgtgttgtgggagctgctgccttggccggcccacttgggaactccttccccaggcgcctgaagctggcccagtttgactacgggaggaagtgctcggaggtcgctcggctgacggagggcatgtcgggccgggagatcgctcagctggccgtgtcctggcaggtgagtcaggctccggcacgtccacccagacgggaccccagctgctgtggagatgctcagttgcgccaggcctgtcccagcaccggtgtcacgcgggagcttctgttgaggggtt

SEQ ID NO: 51 - DNA, 300 bp, synthetic construct
  misc_feature 1..300: note = Synthetic: Fig. 11 ATAD3B
  source 1..300: mol_type = other DNA
tgcctgtcttccggcctccacctcatggtgtggggtccgcggccttggctgcctcacttgggaactccttccccaggcgcctgaagctggcccagtttgactacgggaggaagtgctcggaggtcgctcggctgacggagggcatgtcgggccgggagatcgctcagctggccgtgtcctggcaggtgagtcaggctccggcacgtccacccagacgggaccccagctgctgtggagatgctcagttgcgccaggcctgtcccagcaccggtgtcatgtgggagcttctgttgaggggtt

SEQ ID NO: 52 - DNA, 300 bp, synthetic construct
  misc_feature 1..300: note = Synthetic: Fig. 11 ATAD3C
  source 1..300: mol_type = other DNA
tgcctgtcttccggcctccacctcgtggtgtgggagctgctgccttggccggcccacttgggaactccttccccaggcgtctgaagctggcccagtttgactacgggaggaagtgcttagagatcgctcggctgacagagggcatgtcatgccggaagatcgcacagctggccgtgtcctggcaggtgagtcaggctcgggtgcacccacccagatggaagcccagctgctgtgcagatgcttggttgcgccaggcctgtcccagcaccggtgtcacgtgggagcttctgttgaggggtt

SEQ ID NO: 53 - DNA, 75 bp, synthetic construct
  misc_feature 1..75: note = Synthetic: Fig. 12B
  source 1..75: mol_type = other DNA
gctcggctgacggagggcatgtcgggccgggagatcgctcagctggccgtgtcctggcaggtgagtcaggctccg

SEQ ID NO: 54 - DNA, 49 bp, synthetic construct
  misc_feature 1..49: note = Synthetic: Fig. 13 Clone 5
  source 1..49: mol_type = other DNA
tgacggagggcatgtcggggctggaaaaccnttcactggncctggcctg

SEQ ID NO: 55 - DNA, 49 bp, synthetic construct
  misc_feature 1..49: note = Synthetic: Fig. 13 Clone 6
  source 1..49: mol_type = other DNA
tgacggagggcatatcggtccggtggacctgtcctgtggggtgatcctg

SEQ ID NO: 56 - DNA, 49 bp, synthetic construct
  misc_feature 1..49: note = Synthetic: Fig. 13 Clone 8
  source 1..49: mol_type = other DNA
tgacggagggcatgtcgtcatgtcgtcggagatcgctcagctggccgtg

SEQ ID NO: 57 - DNA, 49 bp, synthetic construct
  misc_feature 1..49: note = Synthetic: Fig. 13 Clone 13
  source 1..49: mol_type = other DNA
tgacggagggcatgtcgggcctggaaaacccttcactgggcctggcctg

SEQ ID NO: 58 - DNA, 49 bp, synthetic construct
  misc_feature 1..49: note = Synthetic: Fig. 13 Clone 15
  source 1..49: mol_type = other DNA
tgacggagggcatgtcgggtgggaaaatccttcacttggcctggtccgg

SEQ ID NO: 59 - DNA, 50 bp, synthetic construct
  misc_feature 1..50: note = Synthetic: Fig. 13 Clone 16
  source 1..50: mol_type = other DNA
tgacggagggcatgtcgnngggagatcgctcagctggccgtgtcctggca

SEQ ID NO: 60 - DNA, 75 bp, synthetic construct
  misc_feature 1..75: note = Synthetic: Fig. 18B
  source 1..75: mol_type = other DNA
ctgtatgggccaccaggcaccgggaagacgctgtttgccaaggtgagagcgcctggctgaacaggtgggccaggg

SEQ ID NO: 61 - DNA, 49 bp, synthetic construct
  misc_feature 1..49: note = Synthetic: Fig. 13 Clones 1, 3, 4, 7, 9, 10, 11, 12, 14, 17, 18, 19, 20, and 21
  source 1..49: mol_type = other DNA
tgacggagggcatgtcgggccgggagatcgctcagctggccgtgtcctg

SEQ ID NO: 62 - DNA, 28 bp, synthetic construct
  misc_feature 1..28: note = Synthetic: Fig. 18A
  source 1..28: mol_type = other DNA
gcaccgggaagacgctgtttgccaaggt

(19) United States
(12) Patent Application Publication
(10) Pub. No.: US 2024/0197914 A1
(43) Pub. Date: Jun. 20, 2024

(54) VARIANT-SPECIFIC EXOGENOUS DNA TEMPLATE-FREE CORRECTION OF PATHOGENIC VARIANTS

(71) Applicants: Oklahoma Medical Research Foundation, Oklahoma City, OK (US); Hadasit Medical Research Services and Development Ltd., Jerusalem (IL)

(72) Inventors: Wan Hee Yoon, Edmond, OK (US); Tamar Harel, Nevo Beitar (IL)

(21) Appl. No.: 18/390,880

(22) Filed: Dec. 20, 2023

Related U.S. Application Data
(60) Provisional application No. 63/476,337, filed on Dec. 20, 2022.

(51) Int. Cl. A61K 48/00; A61P 43/00; C12N 9/22; G16H 20/10

(52) U.S. Cl. CPC A61K 48/005; A61K 48/0025; A61P 43/00; C12N 9/22; G16H 20/10

(57) ABSTRACT

Provided herein are compositions and methods for introducing a genetic change in the genome of a cell with an exogenous DNA template-free Cas-based genome editing system comprising: (i) identifying a target genomic sequence of the genome comprising 88% or higher homology with one or more paralogs or pseudogenes that differ by one or more nucleotides; wherein the homologous region in the one or more paralogs or pseudogenes having a desired nucleotide sequence for transfer to the target genomic sequence after a double-strand break (DSB) by Cas-based genome editing; (ii) introducing into the cell a variant-specific sgRNA to cause a unidirectional transfer of genomic DNA from the homologous DNA sequences to the target genomic sequence, and (iii) contacting the genome of the cell with the variant-specific sgRNA and a Cas-based genome editing system, thereby introducing the genetic conversion without the exogenous DNA template.

### CROSS-REFERENCE TO RELATED APPLICATIONS

This application claims priority to U.S. Provisional Application Ser. No. 63/476,337, filed Dec. 20, 2022, the entire contents of which are incorporated herein by reference.

### STATEMENT OF FEDERALLY FUNDED RESEARCH

This invention was made with government support under R01NS121298 and P20GM103636 awarded by the National Institutes of Health. The government has certain rights in the invention.

### TECHNICAL FIELD OF THE INVENTION

The present invention relates in general to the field of the correction of pathogenic gene variants, are more particularly, to variant-specific exogenous DNA template-free correction of pathogenic variants of genes.

### INCORPORATION-BY-REFERENCE OF MATERIALS FILED ON COMPACT DISC

The present application includes a Sequence Listing which has been submitted in electronically in .XML format via EFS-Web and is hereby incorporated by reference in its entirety. Said .XML copy, created on Dec. 20, 2023 is named “OMRF1034.XML” and is 82,918 byes in size.

### BACKGROUND OF THE INVENTION

Without limiting the scope of the invention, its background is described in connection with pathogenic gene variants and editing of the same.

Recent advances in genome editing, including Clustered Regularly Interspaced Short Palindromic Repeats (CRISPR)/Cas9, offer unprecedented opportunities for gene therapy for genetic diseases. CRISPR/Cas9 is an RNA-guided DNA endonuclease system targeting a specific genomic DNA complementary to a single-stranded guide RNA (sgRNA). Cas9 nuclease produces double-strand breaks (DSBs) at target sites of sgRNA, which in most cases, activates the error-prone nonhomologous end joining (NHEJ) pathway, leading to insertions or deletions (indels) at the target sites. At lower frequencies, the DSBs engage the homology-directed repair (HDR) if a homologous DNA template is provided. Hence, gene corrections for pathogenic variants typically require an exogenous homologous DNA template together with sgRNA/Cas.

One such method is WO2019118949A1, filed by Shen, et al., entitled, “Systems and methods for predicting repair outcomes in genetic engineering”. These applicants are said to teach introducing a desired genetic change in a nucleotide sequence using a double-strand break (DSB)-inducing genome editing system, the method is said to comprise: identifying one or more available cut sites in a nucleotide sequence; analyzing the nucleotide sequence and available cut sites with a computational model to identify the optimal cut site for introducing the desired genetic change into the nucleotide sequence; and contacting the nucleotide sequence with a DSB-inducing genome editing system, thereby introducing the desired genetic change in the nucleotide sequence at the cut site. However, this method led to a significant number of indels.

### SUMMARY OF THE INVENTION

As embodied and broadly described herein, an aspect of the present disclosure relates to a method of introducing a genetic change in the genome of a cell with an exogenous DNA template-free Cas-based genome editing system comprising: identifying a target genomic sequence of the genome comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; introducing into the cell a variant-specific sgRNA that directs gene editing by gene conversion and nonallelic homologous recombination (NAHR) to cause a unidirectional transfer of genomic DNA from the homologous DNA sequences to the target genomic sequence, wherein the gene conversion or NAHR involves a double stranded break; and contacting the genome of the cell with the variant-specific sgRNA and a Cas-based genome editing system, thereby introducing the genetic conversion without the exogenous DNA template. In one aspect, the method of correcting a genetic error in the genome of a cell is in vivo or ex vivo. In another aspect, the genetic change restores the function of a gene. In another aspect, the genetic change corrects a disease-causing mutation. In another aspect, the pathogenic variants are selected from ATAD3A, ATAD3B, Survival of Motor Neuron 1 (SMN1), SMN2, CYP2D6/7, FCGR3A, HBB, HBD, HBG1/2, KRT86/81/83, KRT6B/C/A, HBA2/1, CLCNKA, CLCNKB, KRT14/16/17 or any genes with 88% or higher homology with one or more paralogs or pseudogenes. In another aspect, the target is not a region with microhomology or microduplication. In another aspect, a RNA-guided DNA endonuclease is selected from the group consisting of Cas9, Cas12a, Cas12b, Cas12c, Cas12d, Cas12e, Cas12h, Cas13a, Cas13b, Cas13c, Cpf1, and MAD7, or homologs, orthologs, or paralogs thereof. In another aspect, the sgRNA comprises a variant-specific sgRNA that targets a pathogenic variant immediately adjacent a protospacer adjacent motif (PAM) sequence. In another aspect, the Cas-based genome editing system comprises an RNA-guided DNA endonuclease selected from Streptococcus pyogenes Cas9 or a Staphylococcus aureus Cas9.

As embodied and broadly described herein, an aspect of the present disclosure relates to a method of introducing a genetic change in the genome of a cell with an exogenous DNA template-free system at a target site comprising: obtaining a nucleic acid comprising a variant-specific sgRNA specific for a target genomic sequence comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; introducing into the cell a variant-specific sgRNA that directs gene editing by gene conversion and nonallelic homologous recombination (NAHR) to cause a unidirectional transfer of genomic DNA from the homologous DNA sequences to the target genomic sequence, wherein the gene conversion or NAHR involves a double stranded break; and contacting the genome of the cell with the variant-specific sgRNA and a Cas-based genome editing system, thereby introducing the genetic conversion without an exogenous DNA template. In one aspect, the variant-specific sgRNA comprises the variant-specific sgRNA targets a pathogenic variant immediately adjacent to a protospacer adjacent motif (PAM) sequence. In another aspect, the Cas-based genome editing system comprises an RNA-guided DNA endonuclease selected from Streptococcus pyogenes Cas9 or a Staphylococcus aureus Cas9.

As embodied and broadly described herein, an aspect of the present disclosure relates to a method of making a variant-specific single guide RNA for exogenous DNA template-free gene editing comprising: (i) identifying a target genomic sequence of the genome comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; (ii) analyzing the nucleotide sequence and cut site with a computational model to identify a sequence for the variant-specific single guide RNA; (ii) synthesizing variant-specific single guide RNA, wherein the variant-specific sgRNA in the presence of Cas-based genome editing enzymes edits the target genomic sequence. In one aspect, the variant-specific sgRNA comprises one or more modifications. In another aspect, the modifications are selected from the group consisting of: nucleoside analogs, chemically modified bases, intercalated bases, modified sugars, and modified phosphate group linkers. In another aspect, the guide RNA further comprises one or more phosphorothioate, 5′-N-phosphporamidite linkages, or both.

As embodied and broadly described herein, an aspect of the present disclosure relates to a method of treating a genetic disease in a subject caused by a genetic error in the genome of one or more cells of the subject by introducing a genetic change in the genome of a cell with an exogenous DNA template-free genome editing system at a target site comprising: introducing into the cell a variant-specific sgRNA that directs gene editing by gene conversion and nonallelic homologous recombination (NAHR) to cause a unidirectional transfer of genomic DNA from the homologous DNA sequences to the target genomic sequence, wherein the gene conversion or NAHR involves a double-stranded break, wherein the variant specific sgRNA is specific for a target genomic sequence comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; and contacting the genome of the cell with the variant-specific sgRNA and a Cas-based genome editing system, thereby introducing the genetic conversion without an exogenous DNA template. In one aspect, the method of correcting the genetic error in the genome of a cell is in vivo or ex vivo. In another aspect, the genetic change restores the function of a gene. In another aspect, the genetic change corrects a disease-causing mutation. In another aspect, the pathogenic variants are selected from ATAD3A, ATAD3B, Survival of Motor Neuron 1 (SMN1), SMN2, CYP2D6/7, FCGR3A, HBB, HBD, HBG1/2, KRT86/81/83, KRT6B/C/A, HBA2/1, CLCNKA, CLCNKB, KRT14/16/17 or any genes with 88% or higher homology with one or more paralogs or pseudogenes. In another aspect, the target site is not a region with microhomology or microduplication. In another aspect, an RNA-guided DNA endonuclease is selected from the group consisting of Cas9, Cas12a, Cas12b, Cas12c, Cas12d, Cas12c, Cas12h, Cas13a, Cas13b, Cas13c, Cpf1, and MAD7, or homologs, orthologs, or paralogs thereof. In another aspect, the variant-specific sgRNA targets a pathogenic variant immediately adjacent to a protospacer adjacent motif (PAM) sequence. In another aspect, the Cas-based genome editing system comprises an RNA-guided DNA endonuclease selected from Streptococcus pyogenes Cas9 or a Staphylococcus aureus Cas9.

As embodied and broadly described herein, an aspect of the present disclosure relates to a single guide RNA identified by the method claimed herein. In one aspect, the guide RNA comprises one or more modifications. In another aspect, the modifications are selected from the group consisting of: nucleoside analogs, chemically modified bases, intercalated bases, modified sugars, and modified phosphate group linkers. In another aspect, the single guide RNA further comprises one or more phosphorothioate, 5′-N-phosphporamidite linkages, or both.

As embodied and broadly described herein, an aspect of the present disclosure relates to a vector comprising a nucleotide sequence encoding one or more guide RNAs claimed herein. As embodied and broadly described herein, an aspect of the present disclosure relates to a host cell comprising a vector encoding one or more guide RNAs claimed herein. As embodied and broadly described herein, an aspect of the present disclosure relates to a Cas-based genome editing system comprising a Cas protein complexed with at least one guide RNA identified by the method of the present invention. In another aspect, the method further comprises an expression vector having at least one expressible nucleotide sequence encoding a Cas protein and at least one other expressible nucleotide sequence encoding a guide RNA, and wherein the single guide RNA is identified by the method claimed herein.

As embodied and broadly described herein, an aspect of the present disclosure relates to a method comprising a computational model for selecting a single guide RNA sequence for use with a Cas-based genome editing system that introduces a genetic change in a genome by gene conversion and nonallelic homologous recombination (NAHR), the method comprising: using a processor to identify a polynucleotide sequence for a variant-specific sgRNA specific for a target genomic sequence comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; and synthesizing the variant-specific sgRNA. In one aspect, the computational model is a neural network model having one or more hidden layers. In another aspect, the computational model is a deep learning computational model. In another aspect, the computational model is trained with experimental data to predict a probability of distribution of indel lengths for any given nucleotide sequence and cut site. In another aspect, the computational model is trained with experimental data to predict a probability of distribution of genotype frequencies for any given nucleotide sequence and cut site. In another aspect, the computational model comprises one or more training modules for evaluating experimental data. In another aspect, the computational model predicts genomic repair outcomes for any given input nucleotide sequence and cut site. In another aspect, the method further comprises the step of identifying the available cut sites comprises identifying one or more protospacer adjacent motif (PAM) sequences. In another aspect, the computational model is at least one of: a deep learning computational model; a neural network model having one or more hidden layers; is trained with experimental data to predict the probability of distribution of indel lengths for any given nucleotide sequence and cut site; is trained with experimental data to predict the probability of distribution of genotype frequencies for any given nucleotide sequence and cut site; comprises one or more training modules for evaluating experimental data; or predicts genomic repair outcomes for any given input nucleotide sequence and cut site.

As embodied and broadly described herein, an aspect of the present disclosure relates to a method of introducing a genetic change in the genome of a cell with an exogenous DNA template-free Cas-based genome editing system comprising: (i) selecting a single guide RNA (sgRNA) for use with a Cas-based genome editing system capable of introducing a genetic change into a nucleotide sequence of a target genomic location; (ii) identifying a target genomic sequence of the genome comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; (iii) introducing into the cell the variant-specific sgRNA that directs gene editing by gene conversion and nonallelic homologous recombination (NAHR) to cause a unidirectional transfer of genomic DNA from the homologous DNA sequences to the target genomic sequence, wherein the gene conversion or NAHR involves a double-stranded break; and (iv) contacting the genome of the cell with the variant-specific sgRNA and a Cas-based genome editing system, thereby introducing the genetic conversion without an exogenous DNA template.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D show delivery of variant-specific Cas9-RNP without a donor DNA template shows high-yield gene correction for the heterozygous ATAD3A variant. (FIG. 1A) A schematic for the generation of patient-derived iPSCs carrying the de novo ATAD3A variant (c.1582C>T: p.Arg528Trp) and delivery of the variant-specific Cas9-RNP without a donor template. (SEQ ID NOS: 36, 37 and 38). (FIG. 1B) A schematic of the variant-specific sgRNA targeting the heterozygous variant (c.1582C>T) in exon 15 of the ATAD3A gene (sgRNA-RW). The variant-specific sgRNA is shown (blue color). The letter GGG (cyan) indicates PAM sequence. The blue bar indicates the genomic PCR region for subsequent genomic analysis. (SEQ ID NO:39 in each of C7, C10, C27, and C30, right and left) (FIG. 1C) Sanger chromatograms of iPSCs including C7, C10, C27, and C30 lines before and after the variant-specific Cas9-RNP delivery. Arrowheads with red box indicate the heterozygous c.1582C>T mutations in the patient-derived iPSCs. The blue boxes show their correction after the Cas9-RNP delivery. The arrowheads with dotted lines indicate Cas9 cutting sites. The red underlines show the sgRNA targeting sequences. The cyan dots indicate the PAM sequences. (FIG. 1D) ICE analysis of the Sanger chromatogram from the patient-derived iPSC (C7) after delivering the variant-specific Cas9-RNP shows an increase in C allele (91%) of the PCR products. Error bars indicate SEM (n=3). P values were calculated using Student's t-test (***P<0.001).

FIGS. 2A and 2B shows amplicon NGS confirmed efficient gene correction of the heterozygous ATAD3A variant in iPSCs after the variant-specific RNP delivery. (FIG. 2A) A schematic of the heterozygous c.1582C>T variant in ATAD3A exon 15 and the variant-specific Cas9-RNP induced DSB (left) and observed outcomes (right). Five types of amplicons were observed; uncorrected c.1582 T allele, corrected/WT c.1582 C allele, uncorrected T allele with indels, corrected (or WT) C allele with indels, and deletions covering the c.1582 locus. (FIG. 2B) Amplicon NGS results show allele frequencies for ATAD3A c.1582 C allele (blue), ATAD3A c.1582 T allele (red), c.1582 C allele with indels (black), c.1582 T allele with indels (yellow), and deletions covering the c.1582 locus (grey) in C7, C10, C27, and C30 iPSC lines before and after the variant-specific Cas9-RNP delivery.

FIGS. 3A to 3G show the subclonal selection of the variant-specific Cas9-RNP electroporated iPSCs shows high yield gene-correction in the absence of an exogenous template. (FIG. 3A) An illustration of subclonal selection after delivering the variant-specific Cas9-RNP to the patient-derived iPSCs (C7). Twenty subclones were selected and ICE analyses were performed to identify subclones with gene corrections. Additional NGS was performed to detect large indels in the ATAD3 gene, copy number variation, as well as chromosomal integrity. (FIG. 3B) Representative Sanger chromatograms of gene-corrected and indel-harboring iPSC subclones (left). The arrowhead indicates a gene correction for the heterozygous ATAD3A variant (c.1582C>T) in subclone #20 (SC20). The presence of 1 bp insertion resulted in overlapping sequencing peaks in subclone #5 (SC5). The red underlines indicate the target sequences of the variant-specific sgRNA. Cyan dots indicate PAM sequences. The pie chart shows the gene-correction efficiency of the variant-specific Cas9-RNP electroporation (right). 70% of subclones (n=14) showed the corrected variants (c.1582 C) in the ATAD3A gene (n=20). (SEQ ID NOS:40 and 41) (FIG. 3C-FIG. 3G) Oxygen consumption rates of patient-derived iPSCs (C7) and gene-corrected (SC20) iPSCs. Nine replicates of each iPSC were quantified. Error bars indicate SEM. P values were calculated using Student's t-test (***P<0.001. N.S. indicates not statistically significant).

FIGS. 4A and 4B show gene-corrected iPSCs show intact genomic structure. (FIG. 4A) Integrative Genomics Viewer (IGV) snapshots of the exon 15 in ATAD3A and the homologous regions in ATAD3B and ATAD3C in the patient-derived iPSCs (C7) and the gene-corrected iPSCs (SC20). Indels were not detected in the proximal genomic region of the variant-specific Cas9-RNP target site in the gene-corrected iPSC (SC20). (FIG. 4B) A read depth Manhattan plot across the human chromosome 1 in the patient-derived iPSCs (C7) and the gene-corrected iPSCs (SC20). There is no difference in the overall copy number after the gene correction.

FIGS. 5A to 5C show the variant-specific Cas9-RNP delivery leads to correction for the pathogenic ATAD3A allele in neural progenitor cells without a donor template. (FIG. 5A) Sanger chromatograms of C7 and C27 iPSC-derived NPCs before and after the variant-specific Cas9-RNP delivery. Arrowheads with red box indicate the heterozygous c.1582C>T variant in the patient iPSC-derived NPCs. The blue boxes show the c.1582T to C correction after the Cas9-RNP delivery. The arrowheads with dotted lines indicate Cas9 cutting sites. The red underlines show the sgRNA targeting sequences. Cyan dots indicate the PAM sequences. (SEQ ID NO: 39 both C7 and C27 right and left) (FIG. 5B) ICE analysis for Sanger chromatograms of NPCs (C7 and C27) before and after delivering the variant-specific Cas9-RNP (n=2). (FIG. 5C) NGS results for the ATAD3A c.1582C>T locus in C27 NPCs. The pie charts show allele frequencies for ATAD3A c.1582 C allele (blue), ATAD3A c.1582 T allele (red), c.1582 C allele with indels (black), c.1582 T allele with indels (yellow), and deletions covering the c.1582 locus (grey) before and after the variant-specific Cas9-RNP delivery.

FIGS. 6A to 6D show delivery of the variant-specific Cas9-RNP targeting the additional heterozygous variant in ATAD3A results in efficient gene correction without a donor DNA template. (FIG. 6A) A schematic of the variant-specific sgRNA targeting the additional pathogenic heterozygous variant (c.1076C>T: p.Thr359Met) in exon 10 of the ATAD3A gene. The variant-specific sgRNA (-strand, sgRNA-TM) is shown (bold). The letter CGG (underlined, -strand) indicates a PAM sequence. The map below the sequences indicates the genomic PCR region for subsequent genomic analysis. (SEQ ID NO: 42, 43, and 44). (FIG. 6B) Sanger chromatograms of TM C-9 and TM C-21 iPSCs before and after the variant-specific Cas9- RNP delivery. Arrowheads with red box indicate the heterozygous c.1076C>T variant in the patient-derived iPSCs. Arrowheads (blue box) shows its correction after the Cas9-RNP delivery. The arrowheads with dotted lines indicate the Cas9 cutting sites. The red underlines show the sgRNA targeting sequences. Cyan dots indicate the PAM sequences. (SEQ ID NO: 45 and 46). (FIG. 6C) ICE analysis of the Sanger chromatogram from the TM C21 iPSCs after delivering the variant-specific Cas9-RNP shows an increase in C allele (86%) of the PCR products. (FIG. 6D) Amplicon NGS results show the allele frequencies for ATAD3A c.1076 C allele (blue), ATAD3A c.1076 T allele (red), c.1076 C allele with indels (black), c.1076 T allele with indels (yellow), and deletions covering the c.1076 locus (grey) before and after the variant-specific Cas9-RNP delivery. Error bars indicate SEM (n=2). P values were calculated using Student's t-test (***P<0.001, **P<0.01).

FIGS. 7A and 7B show Cas9-RNP targeting the wildtype ATAD3A and ATAD3B leads to both interallelic and interlocus gene conversion. (FIG. 7A) A schematic for sgRNA-WT that targets both wildtype ATAD3A c.1582 C and its homologous region in ATAD3B. The sgRNA-WT is shown (bold). This sgRNA targets one copy of the wildtype ATAD3A allele and both ATAD3B alleles (left bottom). Schematics for inter-allelic gene conversion between ATAD3A alleles (top right) and inter-locus gene conversions (NAHR) (right bottom). PAM sequence is underlined. (SEQ ID NO: 47, 48, and 49). (FIG. 7B) Amplicon NGS results show allele frequencies for ATAD3A c.1582 (left) and its homologous region in ATAD3B (right). Pie charts show C allele (blue), T allele (red), C allele with indels (black), T allele with indels (yellow), and deletions covering ATAD3A c.1582 locus or its homologous region in ATAD3B (grey) in iPSC lines (C7) before and after the sgRNA-WT Cas9-RNP delivery.

FIGS. 8A to 8G show that RAD51, BRCA1, BRCA2, and CtIP are required for the template-free gene correction in ATAD3A c.1582C>T variant. (FIG. 8A) Schematics of the RAD51-dependent HR and the required proteins in each step. (FIG. 8B-FIG. 8D) ICE analyses show differences in nucleotide contribution at ATAD3A c.1582 following treatment of the variant-specific Cas9-RNP either alone or in combination with siRNAs targeting (FIG. 8B) RAD51, (FIG. 8C) BRCA1, BRCA2, and (FIG. 8D) CHIP. (FIG. 8E-FIG. 8G) Amplicon NGS reveals allele frequencies of ATAD3A c.1582 locus after the variant-specific Cas9-RNP with or without siRNAs targeting (FIG. 8E) RAD51, (FIG. 8F) BRCA1, BRCA2, and (FIG. 8G) CHIP. Error bars indicate SEM (n=3). P values were calculated using Student's t-test (*P<0.05, **P<0.01, ***P<0.001. N.S. indicates not statistically significant).

FIGS. 9A to 9D show the karyotyping of patient-derived iPSCs carrying the heterozygous ATAD3A c.1582C>T variant. (FIG. 9A-FIG. 9B) Fibroblasts obtained from the first individual carrying the de novo ATAD3A c.1582C>T variant were reprogrammed into two iPSC clones: C7 and C10. Both C7 and C10 iPSC lines exhibit normal 46, XX karyotypes. (FIG. 9C-FIG. 9D) iPSC clones C27 and C30 were generated from PBMCs of a second individual carrying the identical heterozygous ATAD3A variant (c.1582C>T), and display normal 46, XX karyotypes.

FIGS. 10A to 10D show flow cytometry analysis of patient-derived iPSCs carrying the heterozygous ATAD3A c.1582C>T variant. Flow cytometry analysis of (FIG. 10A) C7, (FIG. 10B) C10, (FIG. 10C) C27, and (FIG. 10D) C30 iPSC lines using antibodies for OCT4, and SSEA4, hallmarks of pluripotent stem cells.

FIG. 11 shows the alignment of the genomic sequences of the ATAD3A exon 15 and its homologous regions in ATAD3B and ATAD3C. Alignments of genomic sequences from ATAD3A exon 15, ATAD3B exon 15, and ATAD3C Exon 11 using the EMBL-EBI search and sequence analysis tool. The light bar highlights a 222 bp sequence identical between ATAD3A and ATAD3B. The dark bar with uppercase sequences indicates exon 15 of ATAD3A. The c.1582 C allele in ATAD3A is marked in red. The underlined sequence shows the sgRNA-RW targeting site. Bold sequences in ATAD3B and ATAD3C display unique nucleotides in ATAD3B and ATAD3C that differ from ATAD3A (SEQ ID NO: 50, 51 and 52).

FIGS. 12A to 12B shows sanger sequencing and ICE analysis of ATAD3B after delivering ATAD3A c.1582C>T variant-specific Cas9-RNP. (FIG. 12A) Sanger chromatograms for ATAD3B exon 15 from C7, C10, C27, and C30 iPSC lines before (left) and after (right) delivery of the ATAD3A c.1582C>T variant-specific Cas9-RNP. Blue boxes highlight the ATAD3B c.1582C allele in exon 15, which is homologous to the ATAD3A c.1582C allele. (SEQ ID NO:39) (FIG. 12B) ICE analyses for the ATAD3B Sanger chromatograms reveal no indels in ATAD3B exon 15 both before and after the delivery of variant-specific Cas9-RNP. (SEQ ID NO:53 in each of C7, C10, C27 and C30, top and bottom).

FIG. 13 shows Sanger chromatograms of twenty subclones derived from C7 iPSCs electroporated with variant-specific Cas9-RNP. Sanger chromatograms for the ATAD3A exon 15 regions of twenty subclones derived from C7 iPSCs following c.1582C>T variant-specific Cas9 RNP delivery. Red bars highlight the target sequences of the variant-specific sgRNA. Cyan dots indicate the PAM sequences. Black dotted lines indicate the Cas9 cutting site. Blue boxes indicate the correction (c.1582 C) of the ATAD3A c.1582C>T pathogenic variant in 14 subclones, whereas the other 6 subclones exhibit indels (indicated by red boxes). (SEQ ID NO: 61; except Clone 5, SEQ ID NO: 54; Clone 6, SEQ ID NO:55; Clone 8, SEQ ID NO: 56, Clone 13, SEQ ID NO: 57; Clone 15, SEQ ID NO:58, and Clone 16, SEQ ID NO:59).

FIG. 14 shows the chromosomal copy number in the patient-derived and gene-corrected iPSCs. A read-depth Manhattan plot across the human genome for patient-derived (C7, top) and gene-corrected iPSC lines (SC20, bottom). No overall copy number difference is observed after gene correction, except for a 2-copy gain on chromosome 20.

FIGS. 15A and 15B show confocal microscopy of neural progenitor cells derived from patient iPSCs. (FIG. 15A-FIG. 15B) Confocal micrographs display neural progenitor cells (NPCs) derived from two iPSC lines (C7 and C27). Nestin (Red), SOX1 (green in A), and PAX6 (green in B) serve as NPC markers. Nuclei are labeled with DAPI (blue). The scale bar represents 100 μm.

FIGS. 16A and 16B show karyotyping of patient-derived iPSCs carrying the heterozygous ATAD3A c.1076C>T variant. Both (FIG. 16A) TM-C9 and (FIG. 16B) TM-C21 iPSCs exhibit normal 46, XY karyotypes.

FIG. 17 shows the amplicon NGS for ATAD3A exon 10 in TM-C9 iPSCs. The amplicon NGS results show the allele frequencies for the ATAD3A c.1076 C allele (blue), ATAD3A c.1076 T allele (red), c.1076 C allele with indels (black), c.1076 T allele with indels (yellow), and deletions covering the c.1076 locus (grey) both before and after delivery of the variant-specific Cas9-RNP in the TM-C9 iPSC line.

FIGS. 18A to 18B show Sanger sequencing and ICE analysis for ATAD3B following delivery of ATAD3A. c.1076C>T variant-specific Cas9-RNP. (FIG. 18A) Sanger chromatograms for ATAD3B exon 10 from TM-C9 and TM-C21 before (left) and after (right) variant-specific Cas9-RNP delivery. The chromatograms show intact ATAD3B following Cas9-RNP delivery. Blue boxes highlight the ATAD3B c.1076 C allele in exon 10 which is homologous to the ATAD3A c.1076 C allele. (SEQ ID NO:62 in all four boxes). (FIG. 18B) ICE analyses for the ATAD3B Sanger chromatograms reveal no indels in ATAD3B exon 10, both before and after the delivery of variant-specific Cas9-RNP. (SEQ ID NO: 60 for all boxes).

FIGS. 19A to 19D show siRNA-mediated knockdown efficiencies for RAD51, CtIP, BRCA1, and BRCA2 in patient-derived iPSCs. (FIG. 19A-FIG. 19D) Quantitative RT-PCR results show the mRNA levels of (FIG. 19A) RAD51, (FIG. 19B) CtIP, (FIG. 19C) BRCA1, and (FIG. 19D) BRCA2 in C7 iPSCs following siRNA treatment. Error bars indicate SEM. P values were calculated using Student's t-test (**P<0.01, ***P<0.001. N.S. indicates not statistically significant).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The ability to correct pathogenic variants that cause genetic disease traits is highly desirable for developing therapeutic strategies. The present inventors developed a novel strategy for gene correction of a heterozygous pathogenic variant of ATAD3A (ATPase Family AAA Domain Containing 3A) in induced pluripotent stem cells (iPSCs) and neural progenitor cells (NPCs) by gene conversion using Streptococcus pyogenes Cas9 (SpCas9) complexed to a single guide RNA (sgRNA) specifically targeting the mutant allele without providing an exogenous DNA template.

ATAD3A encodes a mitochondrial membrane protein. The inventors discovered that a recurrent de novo variant in ATAD3A (c.1582C>T; p.Arg528Trp) leads to neurological syndrome (Harel-Yoon syndrome; HYOS, MIM #617183), characterized by global developmental delay, hypotonia, axonal neuropathy, optic atrophy, and hypertrophic cardiomyopathy. In addition, it is known that diverse genetic variations, including monoallelic and biallelic variants, deletions, and duplications in ATAD3A, cause neurological diseases. Currently, over 20 genetic variations on the ATAD3A gene have been reported to cause neurological diseases in humans and ATAD3A appears to be the most common gene locus that results in lethal neonatal mitochondrial disease. To date, there is no molecular interventional therapies for the ATAD3A-associated diseases.

Recent advances in genome editing, including Clustered Regularly Interspaced Short Palindromic Repeats (CRISPR)/Cas9, offer unprecedented opportunities for gene therapy for genetic diseases. CRISPR/Cas9 is an RNA-guided DNA endonuclease system targeting specific genomic DNA complementary to a sgRNA. Cas9 nuclease produces double-strand breaks (DSBs) at target sites of sgRNA, which in most cases, activates the error-prone nonhomologous end joining (NHEJ) pathway, leading to insertions or deletions (indels) at the target sites. At lower frequencies, the DSBs engage the homology-directed repair (HDR) if a homologous DNA template is provided.

Hence, gene-corrections for pathogenic variants typically require an exogenous homologous DNA template together with sgRNA/Cas9. Gene conversion is a specific type of homologous recombination (HR) that involves the unidirectional transfer of genetic information: the transfer of DNA from one genomic location without a double-stranded break (DSB) (donor), to another location where DSB occurs (acceptor). The donor sequence can be allelic (the other allele in the homologous chromosome) or nonallelic sequences. Gene conversion by the nonallelic donors including paralogs or pseudogenes is referred to as non-allelic homologous recombination (NAHR). Gene conversion is more frequently observed in genes with paralogs and pseudogenes. Humans have three ATAD3 paralogs: ATAD3A, ATAD3B, and ATAD3C. The genetic architecture (tandem localization) and high homology between the three paralogs make this genomic region prone to NAHR.

Gene editing methods of the prior art suffer from the problem that the design of the constructs leads to significant errors. The present inventors demonstrate an exogenous DNA template-free CRISPR-Cas9-mediated gene correction for pathogenic alleles in the ATAD3A genes by gene conversion and NAHR. Gene conversion is a specific type of homologous recombination (HR) that involves the unidirectional transfer of genomic information. Gene conversion occurs using homologous DNA sequences. Those include homologous allelic genes as well as nonallelic paralogs or pseudogenes. Gene conversion involving nonallelic paralogs is referred to as NAHR. Hence, gene conversion is more frequently observed in the genes with paralogs or pseudogenes. Humans have three ATAD3 paralogs. It is experimentally demonstrated herein that the delivery of a variant-specific sgRNA/Cas9 leads to highly efficient gene correction of the heterozygous pathogenic allele of ATAD3A in both iPSCs (induced pluripotent stem cells) and NPCs (neuronal progenitor cells) despite not providing a template DNA. This strategy can be used for additional pathogenic variants in ATAD3A and expand to pathogenic variants in genes with paralogs in the human genome.

By contrast to the present invention, Shen et al. (WO2019118949A1) demonstrated an exogenous DNA template-free CRISPR-Cas9-mediated gene editing for the correction of pathogenic alleles caused by microduplication and frameshift mutations. This strategy employed a machine learning model, inDelphi that enables prediction of the genotypes and frequencies of the cut-site products caused by NHEJ (non-homologous end joining) and MMEJ (microhomology-mediated end joining), the two pathways in the double-stranded DNA breaks (DSBs) repairs. Shen's group trained inDelphi to design gRNAs to find pathogenic frameshift and microduplication alleles that can be corrected by introducing indels after DSBs by CRISPR/Cas9 editing. Then, Shen experimentally achieved template-free correction of 183 pathogenic human microduplication alleles to wild-type genotypes by removing microduplication (deletion) in >50% of the editing products. Hence, Shen developed a template-free gain-of-function genotypic correction strategy by using MMEJ and/or NHEJ repair mechanisms.

These two strategies are fundamentally different because the molecular mechanisms for gene correction are different (gene conversion for the present invention vs. MMEJ, NHEJ by Shen). Importantly, the methods and constructs demonstrated herein involve targeting genes with paralogs or homologous pseudogenes, whereas Shen's method targets pathogenic alleles with microduplication and some frameshift that are predicted by the inDelphi. In addition, it is shown here that the gene correction in iPSCs and NPCs, whereas Shen only showed the correction in human cell lines and fibroblasts.

To avoid the highly error-prone methods of gene editing, the inventors sought to target, as an example, the ATAD3 paralogs to determine whether CRISPR/Cas9-mediated DSBs in pathogenic alleles ATAD3A can result in gene correction in an exogenous DNA template-free Cas-based gene conversion system with a reduced or no error rate. To investigate CRISPR/Cas9-directed correction for pathogenic variants in ATAD3A by gene conversion, the inventors developed two cellular models, including iPSCs and NPCs derived from patient cells carrying the heterozygous ATAD3A variant (c.1582C>T: p.Arg528Trp). For the mutant allele correction, the inventors employed a CRISPR/Cas9-mediated genome editing with a variant-specific sgRNA. The inventors found that the delivery of a variant-specific Cas9 ribonucleoprotein (RNP) alone leads to highly efficient gene correction of the c.1582C>T allele of ATAD3A in both iPSCs and NPCs despite not providing a template DNA. Whole genome sequencing of a gene-corrected iPSC line and the patient iPSC line confirmed that the genome editing of the variant-specific sgRNA/Cas9 did not cause large indels at the genomic locus around the variant in ATAD3A, as well as off-target regions including the two paralogs ATAD3B and ATAD3C. Furthermore, the gene correction in ATAD3A functionally restored normal mitochondrial function and respiration that were impaired in patient iPSCs.

The present inventors demonstrate, for the first time, that a variant-specific sgRNA/Cas9 leads to highly efficient correction for pathogenic variants (using ATAD3A as an example) without an exogenous DNA template in induced pluripotent stem cells (iPSCs) and neural precursor cells (NPCs). The variant-specific sgRNA/Cas9 delivery strategy that targets the pathogenic variant has been shown to specifically target pathogenic alleles, leading to disruption of the mutations by introducing indels by NHEJ (nonhomologous end joining).¹⁻³ This strategy would work on monoallelic diseases (dominant diseases). Other studies showed that gene correction and precise genome editing require an exogenous DNA template such as single-stranded oligodeoxynucleotide (ssODN).

However, none of the studies show correction of the pathogenic alleles by the exogenous DNA template-free variant-specific sgRNA/Cas9 delivery. The difference between the former studies and the present invention is that ATAD3A has two paralogs—ATAD3B and ATAD3C—that are in tandem on chromosome 1. The paralogs are highly homologous: the coding region of ATAD3A has 96% and 93% identities with ATAD3B, and ATAD3C, respectively. Gene conversion, a specific type of homologous recombination, frequently occurs between genes with high homology (>92%).⁶ Hence, ATAD3B and ATAD3C could be used as natural templates for correcting Cas9-mediated DSB (double-strand break) in ATAD3A by gene conversion. Thus, the exogenous DNA template-free variant-specific Cas9 delivery strategy is useful for correcting pathogenic variants in the genes that have highly homologous paralogs or pseudogenes in the human genome. Importantly, a “template-free (no template DNA)” gene conversion system makes gene therapy simpler as sgRNA and Cas9 are the only required components for correcting pathogenic variants. Furthermore, the present invention has the advantage over the variant-specific Cas9-directed allele disruption strategy because the strategy can work on not only monoallelic variants (dominant diseases) but also biallelic variants (recessive diseases) by correcting the pathogenic variants.

In summary, the present invention provides an efficient gene therapy strategy (exogenous DNA template-free and variant-specific Cas9 delivery), using ATAD3A-associated disease as an example, for the targeting and correction of other human diseases caused by monoallelic or biallelic variants in genes having prologs and pseudogenes.

The ability to correct pathogenic variants that cause genetic disease traits is highly desirable for developing therapeutic strategies. The present inventors developed a novel strategy for gene correction of a heterozygous pathogenic variant in ATAD3A (ATPase Family AAA Domain Containing 3A) in induced pluripotent stem cells (iPSCs) and neural progenitor cells (NPCs) by gene conversion using Streptococcus pyogenes Cas9 (SpCas9) complexed to a single guide RNA (sgRNA) specifically targeting the mutant allele without providing an exogenous DNA template.

ATAD3A encodes a mitochondrial membrane protein. The present inventors discovered that a recurrent de novo variant in ATAD3A (c.1582C>T; p.Arg528Trp) leads to neurological syndrome (Harel-Yoon syndrome; HYOS, MIM #617183), characterized by global developmental delay, hypotonia, axonal neuropathy, optic atrophy, and hypertrophic cardiomyopathy. The inventors and others have discovered that diverse genetic variations, including monoallelic and biallelic variants, deletions, and duplications in ATAD3A, cause neurological diseases. Currently, over 20 genetic variations on the ATAD3A gene have been reported to cause neurological diseases in humans and ATAD3A appears to be the most common gene locus that results in lethal neonatal mitochondrial disease. To date, there is no molecular interventional therapies for the ATAD3A-associated diseases.

Recent advances in genome editing, including Clustered Regularly Interspaced Short Palindromic Repeats (CRISPR)/Cas9, offer unprecedented opportunities for gene therapy for genetic diseases. CRISPR/Cas9 is an RNA-guided DNA endonuclease system targeting a specific genomic DNA complementary to a sgRNA. Cas9 nuclease produces double-strand breaks (DSBs) at target sites of the sgRNA, which in most cases, activates the error-prone nonhomologous end joining (NHEJ) pathway, leading to insertions or deletions (indels) at the target sites. At lower frequencies, the DSBs engage the homology-directed repair (HDR) if a homologous DNA template is provided. Hence, gene corrections for pathogenic variants typically require an exogenous homologous DNA template together with sgRNA/Cas9.

Gene conversion is a specific type of homologous recombination (HR) that involves the unidirectional transfer of genetic information: the transfer of DNA from one genomic location without DSB (donor), to another location where DSB occurs (acceptor). The donor sequence can be allelic (the other allele in the homologous chromosome) or nonallelic sequences. Gene conversion by nonallelic donors including paralogs or pseudogenes is referred to as non-allelic homologous recombination (NAHR). Gene conversion is more frequently observed in the genes with paralogs and pseudogenes. Humans have three ATAD3 paralogs: ATAD3A, ATAD3B, and ATAD3C. The genetic architecture (tandem localization) and high homology between the three paralogs make this genomic region prone to NAHR. Hence, the inventors determined whether CRISPR/Cas9-mediated DSBs in pathogenic alleles in ATAD3A result in gene correction in an exogenous DNA template-free manner by gene conversion.

To investigate CRISPR/Cas9-directed correction for pathogenic variants in ATAD3A by gene conversion, the present inventors developed two cellular models, including iPSCs and NPCs derived from patient cells carrying the heterozygous ATAD3A variant (c.1582C>T: p.Arg528Trp). For the mutant allele correction, the present inventors employed a CRISPR/Cas9-mediated genome editing with a variant-specific sgRNA. The present inventors discovered that the delivery of a mutant-specific Cas9 ribonucleoprotein (RNP) alone leads to highly efficient gene correction of the c.1582C>T allele of ATAD3A in both iPSCs and NPCs despite not providing a template DNA. Whole genome sequencing of a gene-corrected iPSC line and the patient iPSC line confirmed that the genome editing of the variant-specific sgRNA/Cas9 did not cause large indels at the genomic locus around the variant in ATAD3A, as well as off-target regions including the two paralogs ATAD3B and ATAD3C. Furthermore, the gene correction in ATAD3A functionally restored normal mitochondrial function and respiration that were impaired in the patient iPSCs.

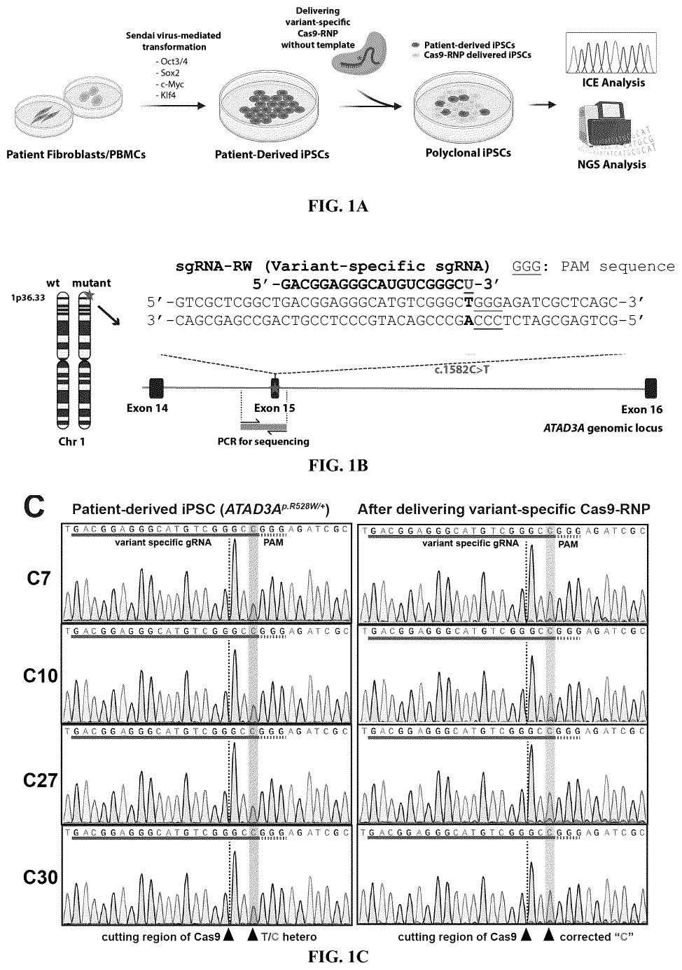

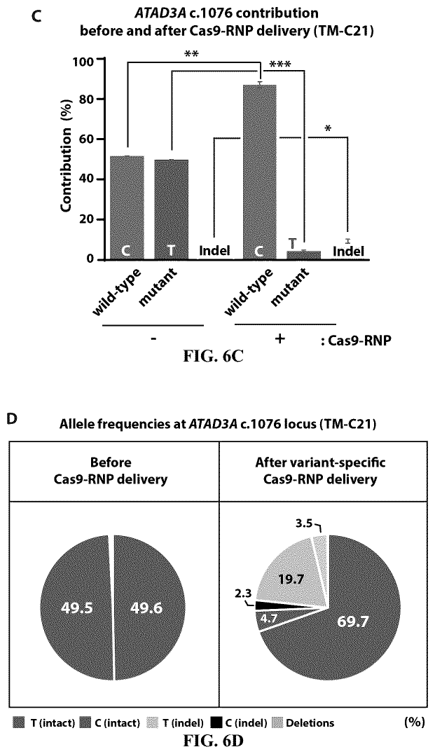

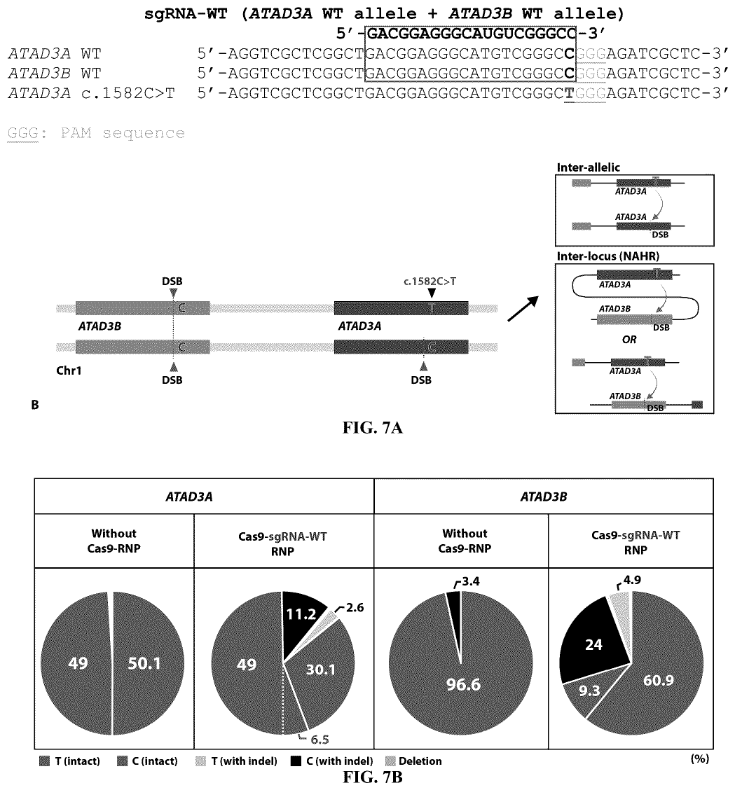

Gene corrected iPS cells without a donor template. The inventors obtained skin fibroblasts carrying the de novo variant (c.1582C>T: p.R528W) in ATAD3A from individual II-2 (female) with HYOS in family 1 (3). Peripheral blood mononuclear cells (PBMCs) were obtained carrying the same de novo variant in ATAD3A from an unrelated individual (female) presenting with HYOS. Using Sendai virus-mediated reprogramming, two iPSC clones were derived from the fibroblasts of the first individual (C7 and C10) and two iPSC clones from the PBMCs of the second individual (C27 and C30) (FIG. 1A). To assess the genomic integrity for the iPSC lines, karyotyping was performed by G-banding, which showed that all iPSC clones exhibited a normal karyotype (46, XX) (FIGS. 9A to 9D). By performing flow cytometry analysis, it was found that approximately 80-90% of all iPSC clones were positive for SSEA4 and OCT4, two hallmarks of human pluripotent stem cells (FIGS. 10A to 10D).

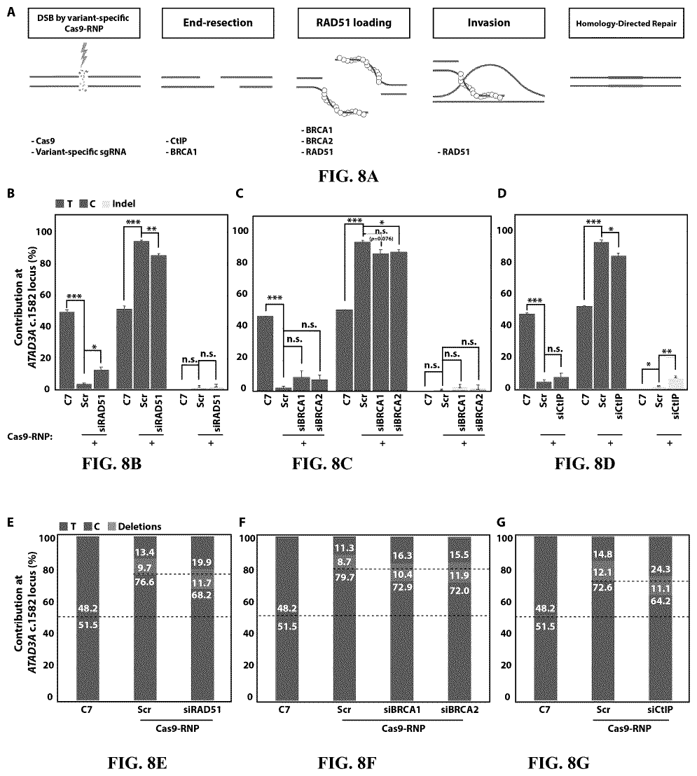

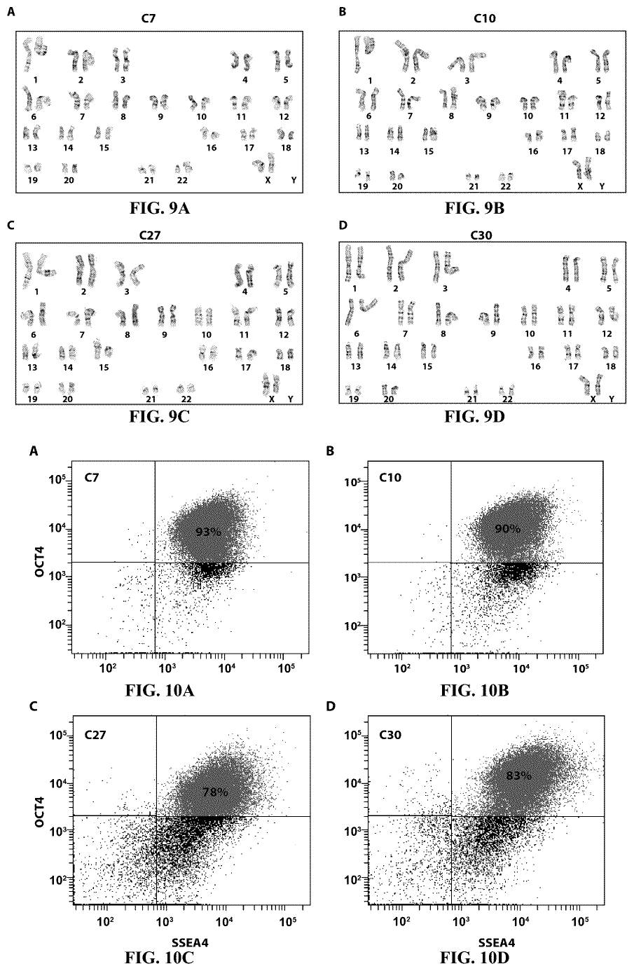

To target the pathogenic variant allele (c.1582C>T) in ATAD3A in C7, the inventors undertook a strategy employing CRISPR-Cas9-mediated mutant allele-specific genome editing (16, 17, 23, 24, 25, 26). Cas9 endonuclease, including Streptococcus pyogenes Cas9 (SpCas9), was reported to tolerate mismatches between sgRNA and target DNA, but not those next to the protospacer adjacent motif (PAM) (13). Hence, a mutant allele-specific guide RNA was designed in which the variant (c.1582C>T) is located next to the PAM sequence (sgRNA-RW) (FIG. 1B). Cas9 was introduced into the iPSCs by electroporation of RNP consisting of sgRNAs and SpCas9 protein (FIG. 1A) (27), which showed less off-site targeting compared to those in vector-mediated Cas9 overexpression (28). The variant-specific Cas9 RNP (sgRNA-RW/spCas9) was electroporated alone into the C7 iPSCs and then performed Sanger sequencing for the genomic PCR product (415 bps) around the target site (FIG. 1B). Inference of CRISPR Editing (ICE) analysis (29) for the Sanger chromatogram of the genomic PCR product revealed ˜90% of the sequences were wild-type (C allele) in the edited C7 iPSCs, despite the absence of a synthetic wild-type DNA template (FIGS. 1C and 1D). An additional iPSC line was examined from the first individual (C10) as well as two iPSC lines (C27 and C30) from the second unrelated individual carrying the same ATAD3A variant (c.1582C>T: p.R528W). The electroporation-mediated delivery of the variant-specific Cas9 RNP led to an efficient T to C correction in all the iPSC lines (FIG. 1C). ATAD3B exon 15 is a potential off-target site because it has 100% identity with ATAD3A exon 15, and the variant-specific gRNA is complementary to ATAD3B sequence except for the last T base (FIG. 11). However, the inventors found no signature for indels or gene editing in the ATAD3B region after delivering the variant-specific Cas9 RNP from the Sanger chromatogram (FIGS. 12A and 12B), indicating that the Cas9 RNP specifically targets the pathogenic allele in ATAD3A. Hence, these data demonstrate that the variant-specific targeting by CRISPR-Cas9 can efficiently correct the pathogenic c.1582C>T variant in ATAD3A without an exogenous DNA template.

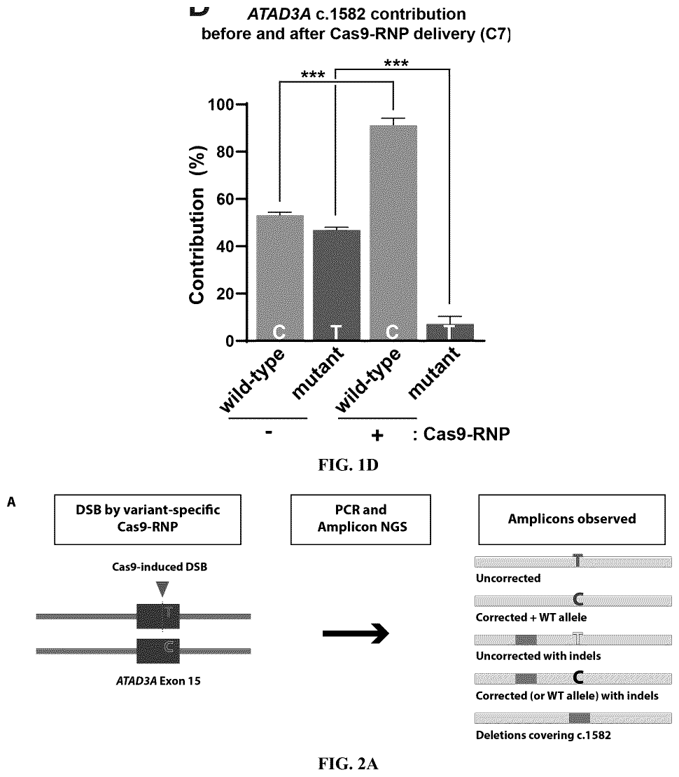

NGS for the PCR amplicon confirmed an efficient gene correction for the pathogenic ATAD3A variant. To assess sequence-level resolution of correction (C/T allele frequency) and indel frequencies, next-generation sequencing (NGS) was performed of the PCR amplicons from C7, C10, C27 and C30 lines before and after the Cas9 RNP delivery (FIG. 2A). It was found that the variant-specific Cas9 RNP (sgRNA-RW) delivery resulted in a significant increase in the C allele (65.2%˜71.8%) and a dramatic decrease in the T allele (6.7%˜13.3%) in all iPSC lines carrying the c.1582C>T variant (FIG. 2B). The analysis revealed that the T allele has more indels than the C allele (FIG. 2B), showing that the variant-specific Cas9 RNP preferentially targets the T allele. The corrected allele frequency is 38%˜53% of the iPSCs having both copies of the wildtype ATAD3A allele (c.1582C) after the Cas9 RNP delivery (Table 1).

TABLE 1
Allele frequencies of ATAD3A c.1582

| iPSC | Cas9- | | | | | | Other | Corrected |
|---|---|---|---|---|---|---|---|---|
| lines | RNP | C (intact) | T (intact) | C (indel) | T (indel) | Deletion | Bases | cell % |
| C7 | — | 50.1% | 49.0% | 0.4% | 0.5% | 0.1% | 0.1% | |
| | sgRNA- | 71.8% | 6.7% | 4.5% | 9.2% | 7.5% | 0.2% | 53.1% |
| | RW | | | | | | | |
| | sgRNA- | 30.1% | 55.5% | 11.2% | 0.4% | 2.6% | 0.2% | |
| | WT | | | | | | | |
| C10 | — | 50.6% | 47.6% | 0.5% | 0.8% | 0.4% | 0.0% | |
| | sgRNA- | 66.4% | 13.3% | 2.6% | 9.7% | 7.8% | 0.3% | 38.6% |
| | RW | | | | | | | |
| C27 | — | 50.1% | 49.0% | 0.4% | 0.4% | 0.1% | 0.1% | |
| | sgRNA- | 69.7% | 7.9% | 3.2% | 11.8% | 7.0% | 0.3% | 46.4% |
| | RW | | | | | | | |
| C30 | — | 50.8% | 48.2% | 0.4% | 0.5% | 0.2% | 0.0% | |
| | sgRNA- | 65.2% | 12.2% | 3.3% | 9.7% | 9.2% | 0.3% | 37.7% |
| | RW | | | | | | | |
| C27 | — | 52.2% | 44.1% | 1.0% | 1.9% | 0.7% | 0.1% | |
| NPC | sgRNA- | 67.3% | 18.1% | 4.4% | 7.7% | 2.5% | 0.0% | 43.5% |
| | RW | | | | | | | |

Hence, the NGS analyses confirmed that a template-free and variant-specific Cas9 RNP delivery achieves efficient correction of the ATAD3A p.R528W variant in multiple iPSC lines derived from unrelated families. In addition, the NGS analysis for ATAD3B amplicons showed a small increase in indel frequency with the C allele (0.9˜3.6%) in the four iPSC lines after delivering the variant-specific Cas9 RNP (Table 2), indicating that the variant-specific Cas9 RNP minimally targets wildtype ATAD3B allele.

TABLE 2
Allele frequencies of ATAD3B c.1582

| | | | | | | | Other |
|---|---|---|---|---|---|---|---|
| iPSC lines | Cas9-RNP | C (intact) | T (intact) | C (indel) | T (indel) | Deletion | Bases |
| C7 | — | 96.6% | 0.0% | 3.4% | 0.0% | 0.0% | 0.0% |
| | sgRNA- | 91.8% | 0.0% | 7.0% | 0.1% | 1.0% | 0.1% |
| | RW | | | | | | |
| | sgRNA- | 60.9% | 9.3% | 24.0% | 0.5% | 4.9% | 0.4% |
| | WT | | | | | | |
| C10 | — | 95.9% | 0.0% | 4.1% | 0.0% | 0.0% | 0.0% |
| | sgRNA- | 94.4% | 0.0% | 5.0% | 0.0% | 0.5% | 0.0% |
| | RW | | | | | | |
| C27 | — | 96.5% | 0.1% | 3.4% | 0.0% | 0.0% | 0.0% |
| | sgRNA- | 94.1% | 0.0% | 5.4% | 0.0% | 0.4% | 0.0% |
| | RW | | | | | | |
| C30 | — | 95.7% | 0.0% | 4.3% | 0.0% | 0.0% | 0.0% |
| | sgRNA- | 92.1% | 0.0% | 6.6% | 0.1% | 1.1% | 0.0% |
| | RW | | | | | | |

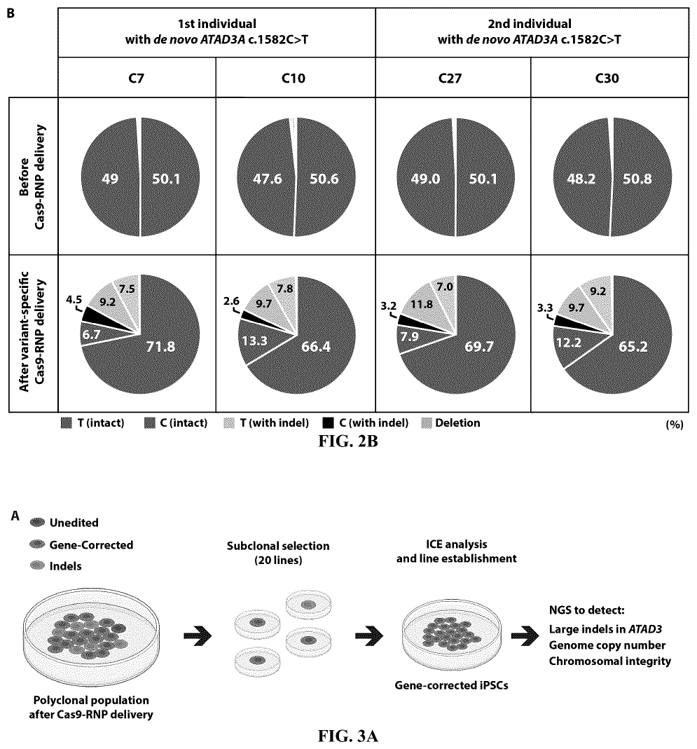

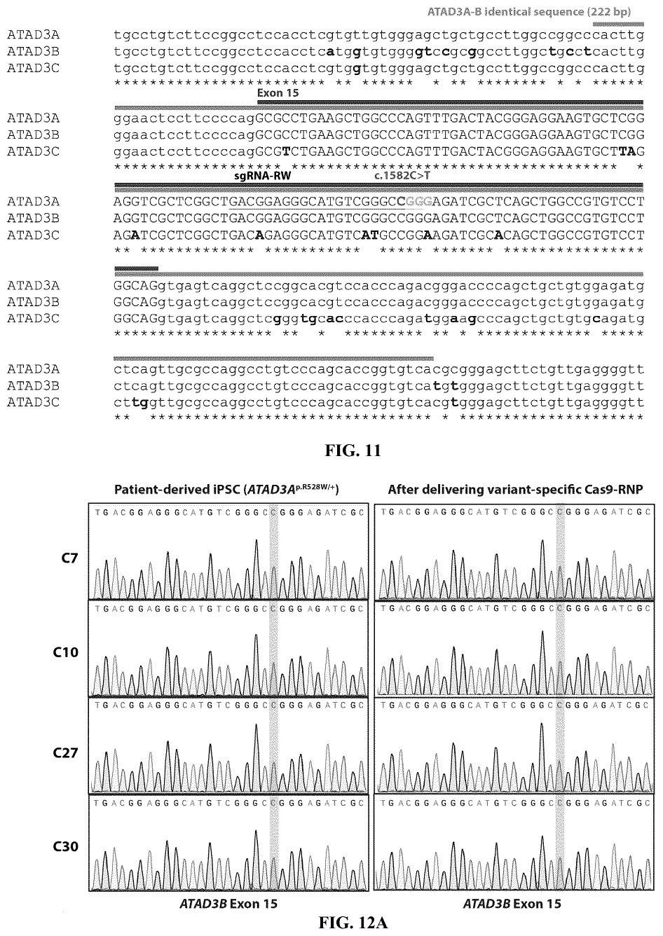

High yield gene-correction in the absence of a template. To further confirm the gene correction of ATAD3A pathogenic variant using the template-free Cas9 approach, subclonal selection was performed and analysis on iPSCs to which the variant- specific Cas9-RNP had been delivered. Twenty clonal colonies were derived from the C7 iPSC line after delivering the variant-specific RNP (FIG. 3A). Sanger sequencing of genomic DNA of these clones showed that 70% of clones (14/20) carried only the wild-type allele, whereas 30% of clones (6/20) harbored indels around the cutting site of the sgRNA (FIG. 3B; FIG. 13). A higher percentage of corrected clones (70%) compared to the expected frequency of corrected cells (53%) from the NGS analysis (Table 1) suggests that the corrected clones outcompete uncorrected ones carrying the pathogenic variant during clonal selection.

Next, the inventors determined the corrected iPSC clones' cellular function associated with mitochondria, as the p.R528W variant was shown to cause defects in mitochondrial maintenance and cristae organization (3). Further, ATAD3A was shown to play a crucial role in mitochondrial respiration (30). To determine the mitochondrial respiration in both patient (C7) and the gene corrected iPSC clones (SC20), a Seahorse metabolic assay was performed that measures cellular respiration and mitochondrial function (31). It was found that the patient iPSCs (C7) exhibited a decrease in both the basal oxygen consumption rate (OCR) and the maximal oxidative capacity compared to those in the gene-corrected iPSCs (SC20) (FIGS. 3C-G). Accordingly, it was found that the ATP-linked respiration is significantly lower in the patient iPSCs (C7) than that in the gene-corrected iPSCs (SC20) (FIG. 3G). Hence, these results demonstrate that the heterozygous p.R528W variant leads to defects in mitochondrial respiration, which was rescued by the gene correction for the p.R528W variant in ATAD3A.

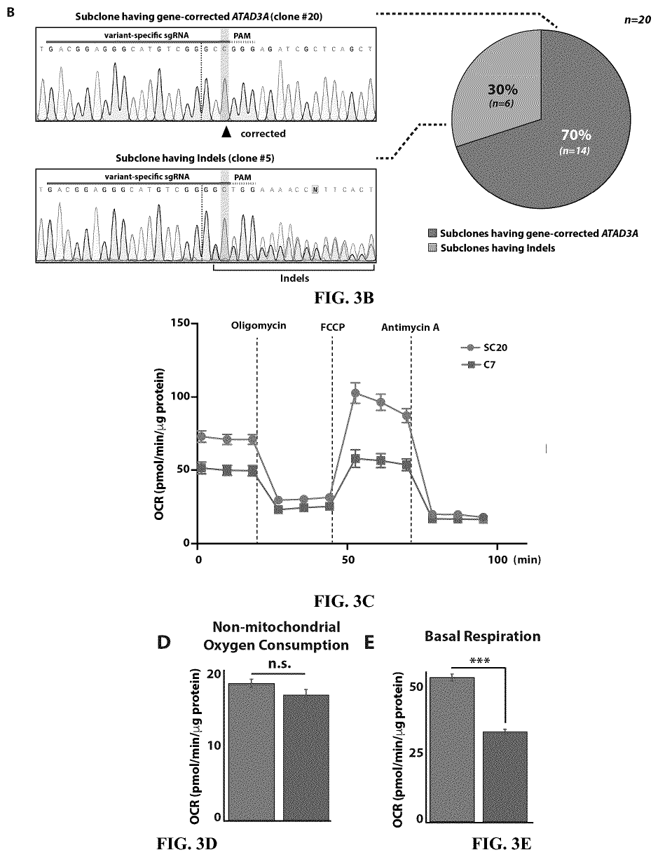

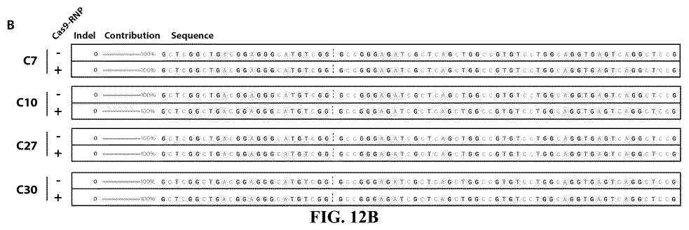

No evidence for on-target indels nor major large-scale aberrations in the genomic architecture. The major on-target effects of CRISPR-Cas9 are thought to be small indels of less than 20 base pairs (bps) (32, 33, 34). CRISPR-Cas9-directed genome editing, however, was also reported to cause large deletions (kilobase-scale) and/or complex genomic rearrangements at the targeted sites in mouse and human cells (35). Thus, genotyping by performing PCR that captures only a small genomic region (415 bps) centered on the Cas9 target site will not determine whether the gene editing resulted in larger structural rearrangements of DNA (>415 bps) that remove primer-binding sites, leading to amplification of only the wild-type allele. To determine whether CRISPR-Cas9-directed DSBs could lead to large deletions at the target site in the ATAD3A genomic locus, whole genome sequencing (WGS) was performed for the patient-derived iPSC (C7) and gene-corrected iPSC (SC20) lines (FIG. 4A). The 30× WGS results showed no sign of large deletions incorporated into the nearby genomic region of the Cas9 target site and chromosome 1 in SC20 (FIGS. 4A-4B). The inventors found a two-copy gain on chromosome 20 in SC20 (FIG. 14), a region previously associated with frequent copy number alterations during iPSC reprogramming (36).

Lack of indels and rearrangements of the target region suggest that the gene correction resulted from HR with the wild-type allelic haplotype of ATAD3A as the template (inter-allelic). If this hypothesis is true, loss of heterozygosity would be found in the flanking regions of the target site in SC20. Nearby present germline heterozygous SNPs (rs9439443, chr1:1,463,337 C>T; rs12032637, chr1:1,465,382 A>G) revealed no difference in their genotypes in C7 and SC20, suggesting that if the recombination did happen, the template length used from the wild-type allelic region of ATAD3A is less than 2045 bps (i.c., the distance between the two SNPs). This is consistent with the previous documentations that gene conversion track is less than 100 bps in mammalian cells (37, 38). Alternatively, gene correction could have occurred through NAHR (inter-locus) with the paralogous genes ATAD3B or ATAD3C. In support of the latter, ATAD3B shares 222 bps of identical sequence with ATAD3A around the target site and maps less than 40 kbps away (FIG. 4A). The corresponding site in ATAD3C diverges in sequence and is thus unlikely to be used as a template (FIG. 11). Hence, the collective data demonstrate that the variant-specific targeting by CRISPR-Cas9 can achieve an efficient correction of the pathogenic variant in ATAD3A by gene conversion: the variant-specific Cas9 RNP causes DSBs at the target sites of the mutant allele, most of which were resolved by gene conversion (interallelic and/or interlocus) leading to gene correction (C allele), but some by NHEJ leading to indels. Alternatively, a nick may have introduced a collapsed fork and a one ended double strand DNA (ocDNA) may be repaired by break-induced replication (BIR) (39, 40).

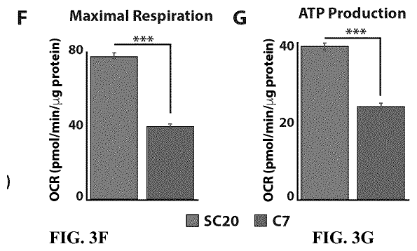

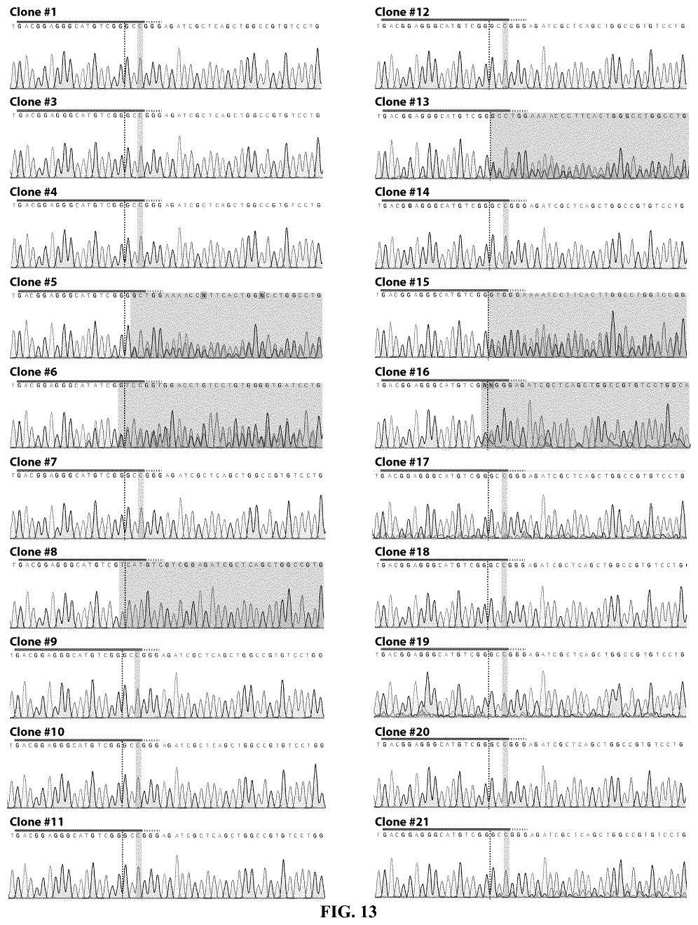

The variant-specific Cas9 RNP enabled correction of the Arg528Trp variant in neural progenitor cells without a donor template. To determine whether the template-free and variant-specific Cas9 RNP delivery can also achieve gene correction for more differentiated cells, the two patient-derived iPSC lines (C7 and C27) were differentiated into neural progenitor cells (NPCs). NPCs have more restricted potential and can be differentiated into neurons, oligodendrocytes, or astrocytes (41). By performing immunocytochemistry, it was shown that the NPCs derived from C7 and C27 are positive for PAX6, SOX1, and Nestin, hallmarks of NPCs (FIGS. 15A and 15B). The variant-specific Cas9 RNP (sgRNA-RW) were electroporated into the C7 and C27-derived NPCs without an exogenous DNA template, and then performed Sanger sequencing for PCR DNA that captured the Cas9 target site. ICE analysis for the Sanger chromatograms of the genomic PCR products from the NPCs revealed 89% in C7 and 76% in C27 of the sequences were wild type (C allele) (FIGS. 5A and 5B). NGS analysis for the C27 NPCs also showed increased allele frequency for C (from 52.2% to 67.3%) and decreased T allele frequency (from 44.1% to 18.1%) (FIG. 5C). Hence, the results demonstrated that the template-free and variant-specific strategy enables correction of the pathogenic variant in ATAD3A in NPCs. It is worth noting that before treating the Cas9 RNP, the C27 NPCs already harbor altered C/T allele frequency and increased indels (1% of C allele reads with indel and 1.9% of T allele reads with indel) (FIG. 5C). This observation is consistent with the previous studies that reported DSBs arising frequently in neural stem and progenitor cells during neurogenesis (42, 43).

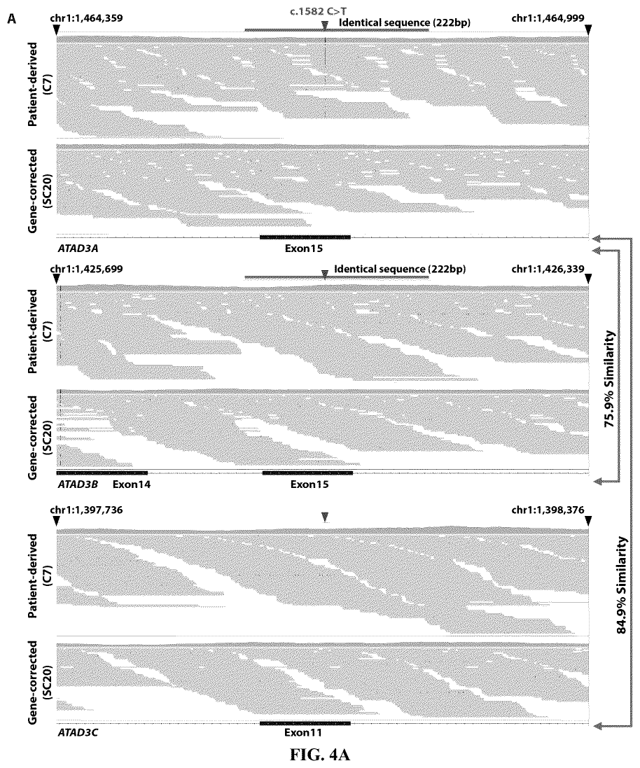

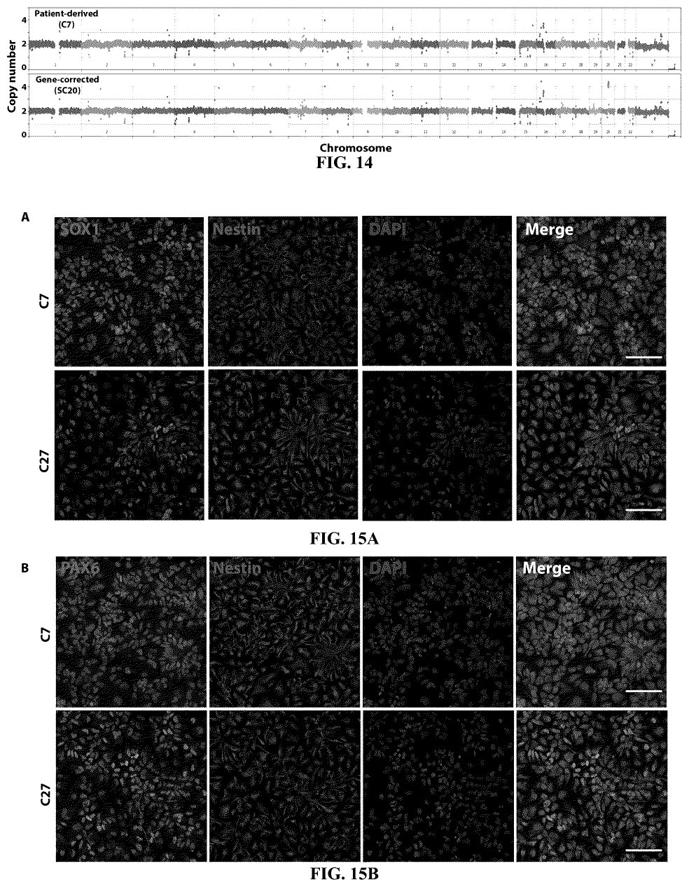

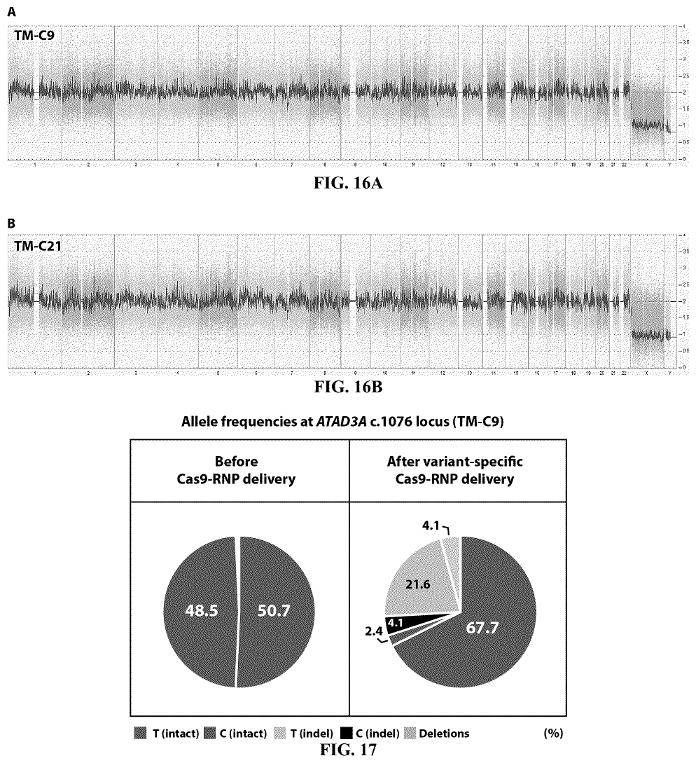

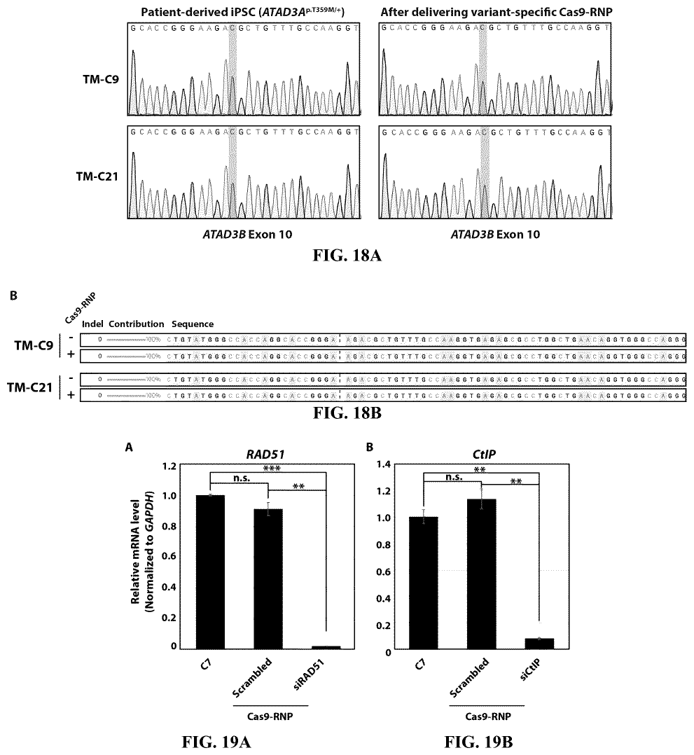

The variant-specific Cas9 RNP enabled correction of an additional pathogenic variant in ATAD3A. To determine whether the variant-specific Cas9 RNP strategy can also achieve gene correction for other pathogenic variants in ATAD3A, the inventors decided to test their gene editing approach for iPSCs, which were derived from HYOS patient fibroblasts carrying biallelic ATAD3A variants—a missense variant (NM_001170535.3; c.1076C>T: p.Thr359Met) in trans to a splicing variant (NM_001170535.3; c.1090-3C>G). The genomic integrity and karyotypes for two iPSC lines (TM-C9 and TM-C21) were confirmed by performing the KaryoStat assay (FIGS. 16A and 16B). The missense variant (c.1076C>T: p.Thr359Met) is predicted to be deleterious by the SIFT algorithm (44). The inventors designed and synthesized a new sgRNA (sgRNA-TM) specifically targeting for the c.1076C>T p.Thr359Met variant (FIG. 6A). It was found that targeting the pathogenic T allele (c.1076T) by treating the variant-specific Cas9 RNP (sgRNA-TM) in both iPSC lines without an exogenous DNA template led to a significant conversion of T to C allele (FIGS. 6B-6C). ICE analysis for the Sanger chromatograms of the genomic PCR products from the TM-C21 line revealed a significant increase in wildtype C allele (86%) (FIG. 6C). In addition, NGS analysis for the TM-C21 and TM-C9 lines also showed increased allele frequency for C (69.7% and 67.7%) and decreased T allele frequency (4.7% and 2.4%) (FIG. 6D and FIG. 17). ATAD3B exon 10 is a potential off-target site because the sgRNA-TM is complementary to ATAD3B sequence except for one nucleotide (C/T). However, the sgRNA-TM-Cas9 RNP delivery did not result in indels or gene editing in the ATAD3B region (FIG. 18A and 18B), indicating that the variant-specific Cas9 RNP (sgRNA-TM) specifically target the pathogenic allele (c.1076C>T: p.Thr359Met) in ATAD3A. Hence, together with the former results for the c.1582C>T: p.Arg528Trp correction (FIGS. 1A to 2D), these data demonstrated that the template-free and allele-specific strategy enables efficient correction of the pathogenic ATAD3A variants.

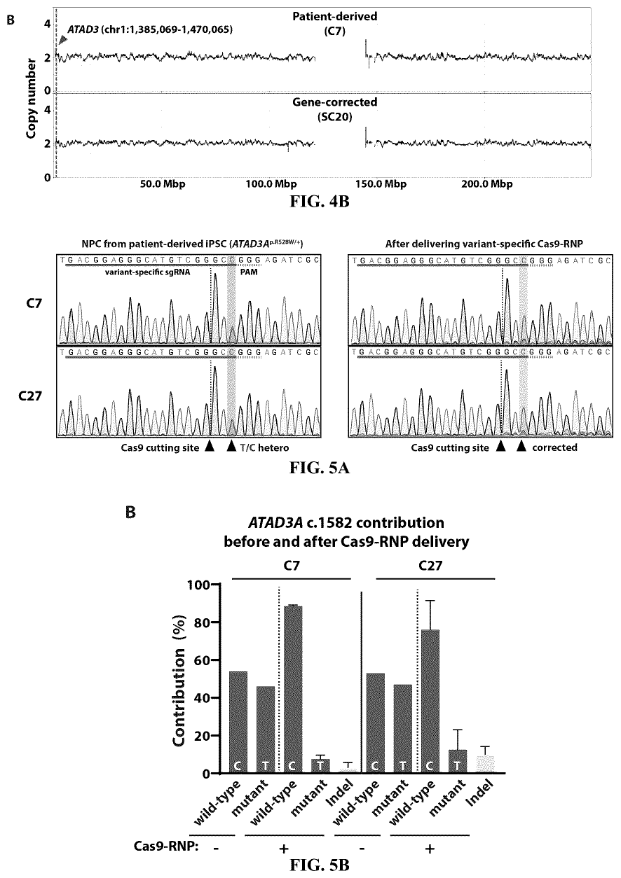

Gene conversion between ATAD3 paralogs. These results (FIGS. 1A to 6D) show that the observed high frequency of correction for the heterozygous variants in ATAD3A is mediated by gene conversion. Gene conversion for the heterozygous c.1582C>T, p.R528W variant can be interallelic (ATAD3A allele in the homologous chromosome) and/or interlocus (non-allelic) (ATAD3B alleles in the same or the other homologous chromosome). The experiments described above cannot distinguish whether the gene conversion was mediated by interallelic, or interlocus, or both interallelic and interlocus recombination, as gene conversions in mammalian cells typically extend less than 100 bps (37, 38) and ATAD3A and ATAD3B have an identical 222 bp region centered around the Cas9 cutting site (FIG. 4A). To answer this question, an experiment was designed with a new sgRNA specifically targeting wild-type c.1582C allele for both ATAD3A and ATAD3B (sgRNA-WT) (FIG. 7A). This sgRNA-Cas9 is expected to generate DSBs in both ATAD3B alleles and a single wildtype ATAD3A allele, leaving the ATAD3A pathogenic allele (c.1582C>T: p.R528W) as the only available template for HR. It was found that electroporation of the wildtype-specific Cas9 RNP (sgRNA-WT) in the C7 iPSC line led to C to T gene conversion in both wildtype ATAD3A and ATAD3B, but at different frequencies (FIG. 7B). NGS analysis showed 6.5% increased T allele frequency for ATAD3A and 9.3% increased T allele frequency for ATAD3B, indicating that the gene conversion occurs through both interallelic and interlocus events (FIG. 7A). The higher (9.3%) gene conversion in ATAD3B may result from two-fold more DSBs in ATAD3B alleles than those in ATAD3A. Different from the efficient correction of ATAD3A pathogenic allele (c.1582C>T: p.R528W) (FIGS. 1A to 1D and 2A and 2B), many DSBs resulted in failed correction with indels (C alleles with indel) (FIG. 7B, black in the pie charts), suggesting that one copy of the c.1582C>T ATAD3A allele was insufficient to provide a template for correction of the DSBs in both ATAD3A and ATAD3B. These data show that the correction of ATAD3A pathogenic allele (c.1582C>T: p.R528W) in FIGS. 1A to 2B results from both interallelic (ATAD3A) and inter-locus (ATAD3B) recombination, and that the higher correction efficiency (65.2%˜71.8% increased C allele) (FIG. 2B) come from gene conversion using three times more substrates (one copy of wildtype ATAD3A and two copies of wildtype ATAD3B).

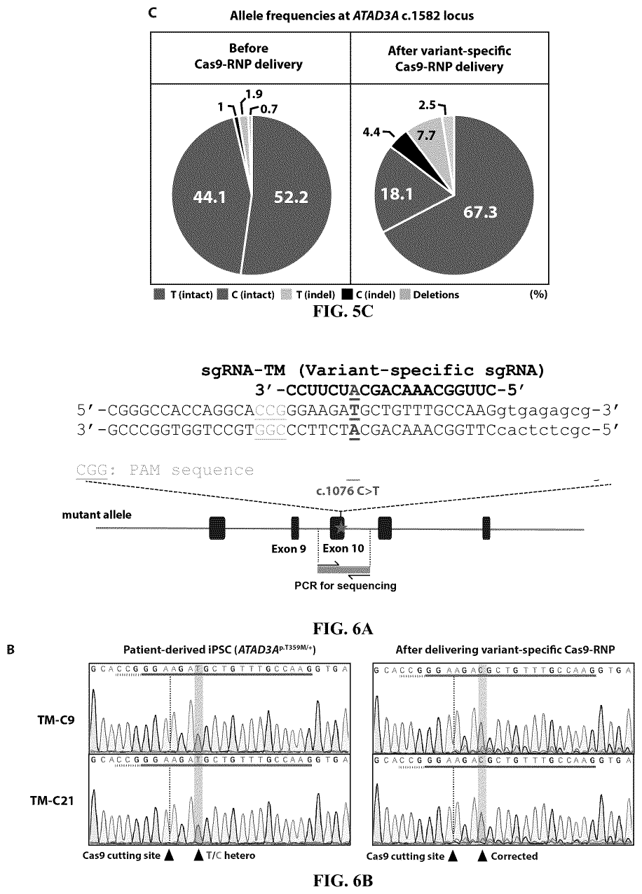

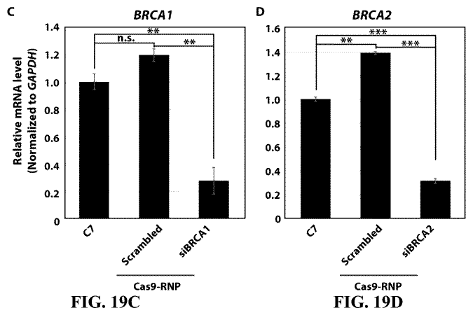

RAD51, BRCA1/2, and CtIP are required for efficient correction of ATAD3A pathogenic variant. Most HR requires RAD51, the ortholog of E. coli RecA, that plays a key role in strand invasion and DNA homology search (45). However, RAD51-independent HR repair also has been reported, including intrachromosomal recombination, break-induced recombination (BIR), single-strand template repair in S. cerevisiae (46, 47, 48) and break-induced telomere synthesis in human cell lines (49). Furthermore, CRISPR-Cas9-mediated single-strand template repair does not require RAD51 in human cells (50). To determine whether the gene correction of the pathogenic ATAD3A variant by the template-free CRISPR-Cas9 editing requires RAD51, siRNA-mediated knockdown was performed for RAD51 in the C7 iPSCs (FIG. 19A) when inducing DSBs in the c.1582C>T (p.R528W) allele by delivering sgRNA-RW/Cas9 RNP. ICE analysis showed that RAD51 knockdown leads to a significant reduction of C allele correction and increased T allele frequency (FIG. 8B). NGS for the amplicon of RAD51 knockdown iPSCs also showed impaired correction (FIG. 8E). Hence, the template-free and variant-specific ATAD3A gene correction requires RAD51.

Next, the inventors determined whether BRCA1 (BRCA1 DNA repair associated) and BRCA2 (BRCA2 DNA repair associated) are required for ATAD3A gene correction, since both proteins play a key role in recruiting RAD51 to DSBs (45). BRCA1 plays a role in two distinct steps: 5′ to 3′ resection of DSBs to generate 3′ ssDNA overhangs by directly interacting with the resection factor CtIP (51, 52) and loading of the RAD51 recombinase into the ssDNA through interacting with PALB2-BRCA2 (53, 54). The inventors determined whether BRCA1, BRCA2, and/or CtIP are required for correcting CRISPR-Cas9-mediated the ATAD3A pathogenic variant. Both ICE and NGS analyses showed that knockdown of all three proteins negatively impact gene correction (FIGS. 8C, 8D, 8F, and 8G; FIGS. 19B-19D). Hence, the results demonstrate that the BRCA1 and CtIP-mediated end resection and BRCA1/2-mediated RAD51 loading are involved in the ATAD3A gene correction.

Gene conversion is a subtype of HR that frequently occurs between paralogs and pseudogenes (19). Three ATAD3 paralogs, including ATAD3A, ATAD3B, and ATAD3C, are located in tandem within an ˜85 kb genomic interval on chromosome 1p36:33 in the human genome (3). This genomic architecture predisposes the ATAD3 genes to be substrates for NAHR during meiosis, resulting in reciprocal CNVs, deletions, and duplication, which lead to neonatal lethal presentations (3, 4, 5, 6, 8). Notably, CRISPR-Cas9-induced DSBs in ATAD3A in mitotic HEK293T cells resulted in gene conversion between ATAD3A and ATAD3B without inducing on-target deletion or duplications (22), showing that CRISPR-Cas9-mediated intentional gene conversion has a therapeutic benefit. Here, the inventors demonstrate that CRISPR-Cas9-induced gene conversion can correct pathogenic variants in ATAD3A in iPSCs and NPCs. They further demonstrate efficient gene correction of the recurrent de novo variant (c.1582C>T, p.R528W) in ATAD3A in iPSCs using SpCas9/allele-specific sgRNA RNP without providing an exogeneous DNA template.

To evaluate the gene correction, both ICE analysis and NGS amplicon sequencing were performed for the patient-derived iPSCs after the RNP delivery and found that 65˜70% of the sequences were wild-type (FIGS. 1A to 1D and 2A and 2B). These results were confirmed in the subclonal iPSCs derived from the RNP-treated patient-derived iPSCs −70% of gene correction for the heterozygous p.R528W variant was observed in the single-cell clones (FIGS. 3A to 3G). CRISPR-Cas9 genome editing could lead to large deletions and genomic rearrangements from a few kilobases to many megabases in mouse and human cells as well as in vivo models, including mouse and zebrafish (35, 55, 56, 57, 58, 59). To determine whether large deletions and genomic rearrangements were present, WGS and genomic analyses were performed for a gene-corrected iPSC line together with the patient-derived iPSC line and did not find any large deletion or genomic rearrangement (FIGS. 4A and 4B). The inventors demonstrated that the template-free, allele-specific Cas9 editing also leads to efficient correction of the c.1582C>T variant in NPCs (FIGS. 5A to 5C) as well as correction of an additional ATAD3A pathogenic variant (c.1076C>T, p.Thr359Met) in iPSCs (FIGS. 6A to 6D). Further, it was found that the correction results from both interallelic and interlocus gene conversion (FIGS. 7A and 7B). Lastly, it was found that the gene correction employs end resection and RAD51-dependent HR mechanism (FIGS. 8A to 8G). Hence, these collective data provide robust evidence that CRIPSR/Cas9-mediated intentional gene conversion can correct the pathogenic variants in ATAD3A.

Since the initial finding of the de novo ATAD3A p.R528W variant as a pathogenic mutation (3), numerous pathogenic alleles at the locus, including both monoallelic and biallelic variants in ATAD3A have been described (4, 5, 6, 7, 8, 9, 10, 11). ATAD3A now appears to be the most common gene locus that results in lethal neonatal mitochondrial disease (8). Recently, the inventors found that missense variants or single nucleotide variants (SNVs) in trans to deletion or frameshift alleles lead to varied severity of phenotypes ranging from neonatal lethality to hypotonia, global developmental delay, learning difficulties, and ataxia (4). Adult heterozygous carriers who harbor one copy of ATAD3A loss-of-function alleles, however, exhibited no substantial health problems, indicating that one intact copy is sufficient for normal human development and physiology (3, 4). Hence, gene correction of one pathogenic missense or SNV allele is expected to be sufficient for restoring biological balance for individuals and human embryos with biallelic variants in ATAD3A. The approach for the gene correction of ATAD3A variants reported in this study could be applied for correcting additional ATAD3A pathogenic variant alleles.

In mammalian cells, DSBs are thought to be mostly repaired by the NHEJ pathway (60), often leading to erroneous correction with indels. NHEJ is more frequently used for DSB repair in part because the NHEJ pathway is speedy and is active in all stages of the cell cycle (60). In contrast, HDR is rarely utilized for DSB repairs, occurring largely during the S and G2 phases of the cell cycle, as the activity of the HR machinery is regulated by cyclin-dependent kinases (CDKs) (61). The HDR machinery requires undamaged sister chromatids or exogenous DNA templates for repairing DSBs. Hence, these features of the HDR pathway make it more useful for introducing precise genetic modifications for CRISPR/Cas9-mediated genome editing. To enhance CRISPR/Cas9-mediated HDR, numerous approaches have been developed including suppression of key NHEJ factors (62), enhancing HDR factors via chemical compounds (63), RAD52 ectopic expression (64), and CtIP fusion to Cas9 (65). Enhancement of precise genome editing can also achieved by using a mutant allele-specific sgRNA. This approach led to a better gene correction yield for dominant disease models and required an exogenous template (16, 17). Unlike the previous reports, using the present invention the inventors found efficient gene correction for iPSCs with the heterozygous ATAD3A p.R528W and p.T359M variants by simply delivering the mutant allele-specific RNP, without an exogenous DNA template.

Initially, it was hypothesized that the gene correction may result from NAHR (interlocus HR) between ATAD3A and ATAD3B, a paralog of ATAD3A, because the genomic structure (tandem localization) of high homology between the paralogs makes this genomic region prone to NAHR (3, 4, 5, 66). However, sgRNA-WT/Cas9 experiments (FIGS. 7A and 7B) showed that HR can occur via both interallelic (between ATAD3A alleles) and interlocus recombination (NAHR) between ATAD3A and ATAD3B. Hence, these results show that higher efficiency of gene correction for the heterozygous variant in ATAD3A results from three endogenous HR templates (one ATAD3A allele and two ATAD3B alleles).

An in-depth analysis of the corrected clonal iPSCs (SC20) genomic region for three paralogs confirms that the corrected genomic region in exon 15 of ATAD3A does not include sequence signatures of ATAD3B or ATAD3C (FIG. 4A), showing that the correction results from HR with wild-type allele of ATAD3A using template of identical sequence of up to 2045 bps (i.c., the distance between the two SNPs in ATAD3A).

By way of explanation, but not a limitation of the present invention, another possible explanation is that the correction may result from NAHR using a short sequence template in ATAD3B (i.c., up to 222 bps of the identical sequence in ATAD3A and ATAD3B). The minimum length of homologous sequence for gene conversion is defined as the Minimal Efficient Processing Segment (MEPS) (19). The rate of gene conversion is directly proportional to the length of the uninterrupted-sequence track in the putatively converted region and the homology between the interacting sequences is always at least 92% and usually >95% (19). The MEPS for meiotic HR in mouse cells is >200 bp (67, 68). The MEPS for meiotic gene conversion in humans is estimated to be in the range of 337-456 bp (69); however, MEPS for mitotic recombination in humans has not been thoroughly studied. Given that both interallelic and interlocus HR (NAHR) can occur between ATAD3A and ATAD3B and the 222 bps sequence identity of ATAD3A and ATAD3B, this study shows that the MEPS for mitotic gene conversion in human cells may be in the rage of ˜200 bps, e.g., 180 to 220, 190 to 210, 195 to 205, or 196, 197, 198, 199, 200, 201, 202, 203, 204, 205, or 200+/−1, 2, 3, 4, 5%.

The delivery of a mutant allele-specific sgRNA/SpCas9 RNP is an effective way for gene correction with the heterozygous pathogenic variant in ATAD3A in iPSCs and NPCs was demonstrated herein. This method can be applied to other types of human cells with additional pathogenic variants in ATAD3A as well as pathogenic variants in the genes having highly homologous paralogs (e.g., Survival of Motor Neuron 1 (SMN1), SMN2, CYP2D6/7, FCGR3A, HBB, HBD, HBG1/2, KRT86/81/83, KRT6B/C/A, HBA2/1, CLCNKA, CLCNKB, KRT14/16/17) to achieve gene correction by intentional gene conversion.

Study Design. The objective of this study was to determine whether intentional gene conversion, induced by CRISPR-Cas9, could correct pathogenic variants in ATAD3A in patient-derived iPSCs and NPCs, and to determine the underlying molecular mechanism. This study used iPSCs derived from individuals diagnosed with HYOS who carry pathogenic variants in ATAD3A. The inventors measured the allele frequency of pathogenic allele both before and after delivering variant-specific Cas9 RNP, by performing ICE analysis from Sanger chromatograms and performing NGS analyses for PCR amplicons of target sites. The numbers of replications varied and is specified in each figure. The study was not blinded.

Cell culture and iPSC generation. This study was approved by an institutional review board (IRB) at Oklahoma Medical Research Foundation, and written informed consent was obtained prior to genetic testing and sample collection. Primary fibroblasts from patient (individual II-2 in family 1) with the ATAD3A c.1582C>T, p.R528W/+variant were obtained in a previous study (3). PBMCs carrying the same de novo ATAD3A variant (c.1582C>T) and fibroblasts carrying biallelic variants (NM_001170535.3; c.1076C>T: p.Thr359Met in trans to a splicing variant, NM_001170535.3; c.1090-3C>G) were collected from individuals with HYOS. Patient fibroblasts and PBMCs were reprogrammed by using CytoTune™ iPS 2.0 Sendai Reprogramming Kit (Thermo Fisher #A16517) according to the manufacturer's guidelines. Briefly, 2.0×10⁵ fibroblasts/PBMCs were transduced with a Sendai virus cocktail encoding hOct3/4, hSox2, hc-Myc and hKlf4. The virus cocktail was removed after 24 hours. After 5 days, cells were detached with Accutase (StemCell Technologies, Cat #07920) and seeded onto hESC-qualified Matrigel (Corning Cat #354277)-coated 10-cm culture plates. After 21 days, iPSC colonies were picked and plated onto Matrigel-coated 24-well plates. Patient-derived iPS cells and gene-corrected iPS cells were cultured in mTeSR1 medium (StemCell Tech #85850) in Matrigel-coated plates at 37° C. in a 5% CO₂ humidified incubator.

CRISPR/Cas9 Design, Electroporation for Cas9-RNP. The variant-specific gRNA was designed with help of Benchling's prediction program as well as TrueGuide™ gRNA design service (Thermo Fisher Scientific) (Table 3). The sgRNA carrying the variant-specific gRNA was synthesized by Synthego (Redwood, California). For gene editing, SpCas9-sgRNA RNP complexes were delivered into iPSCs or NPCs using Neon™ Transfection System (Invitrogen, Waltham, MA). Briefly, 1×10⁵ iPSCs or NPCs in 10 μL Resuspension Buffer R were transfected with 10 pmol of SpyFi Cas9 Nuclease (Aldevron, Cat #9214) and 50 pmol of the sgRNA. For siRNA treatment, 200 pmol siRNAs (Integrated DNA Technologies) were added to 1×10⁵ iPSCs (Table 5). The electroporation protocol for iPSCs was pulse voltage=1,100 V, pulse width=20 ms, pulse number=2, and for NPCs was pulse voltage=1,700 V, pulse width=20 ms, pulse number=2. After electroporation, iPSCs or NPCs were seeded into a well of Matrigel-coated 12-well plates in StemFlex medium (Gibco Cat #A3349401) (iPSCs) or STEMdiff™ Neural Progenitor Medium (StemCell Technologies Cat #05833) supplemented with 10 μM Y-27632. After 2 days of electroporation, cells were detached with Accutase™ (StemCell Technologies, Cat #07920) and followed by genomic DNA purification. For generating single-iPSC clones, SpCas9-sgRNA RNP complexes were delivered into iPSCs using the 4D-Nucleofector™ electroporation system (Lonza, Basel, Switzerland) using Program CA-137. Briefly, 3×10⁵ iPSCs (C7) in 20 μL P3 primary cell solution (Lonza, Cat #V4XP-3032) were nucleofected with 40 pmol of SpyFi Cas9 Nuclease (Aldevron, Cat #9214) and 200 pmol of the sgRNA. Immediately following nucleofection, the cells were seeded at low density onto Matrigel- coated 10-cm plates in StemFlex medium (Gibco Cat #A3349401) supplemented with 10 μM Y-27632 (StemCell Technologies Cat #72302). Clonal colonies were manually picked 10 days after nucleofection, and re-adapted to mTeSR1 medium for expansion and routine maintenance.

TABLE 3
List of single-guided RNAs and
corresponding sequences

| | | | SEQ ID |
|---|---|---|---|
| sgRNA | Sequences | PAM | NO: |
| sgRNA-RW | 5′-GACGGAGGGCAUGUCGGGCU-3′ | GGG | 1 |
| sgRNA-WT | 5′-GACGGAGGGCAUGUCGGGCC-3′ | GGG | 2 |
| sgRNA-TM | 5′-CUUGGCAAACAGCAUCUUCC-3′ | CGG | 3 |

TABLE 4
Primers utilized for the amplification of
ATAD3A and ATAD3B (F-forward, R-reverse).

| Primer | Sequences | Target |
|---|---|---|
| ATAD3A | 5′-CCTGCAGCCACTCCCTGCTC-3′ | ATAD3A |
| -RW_F4 | (SEQ ID NO: 4) | Exon15 |
| ATAD3A | 5′-CCCTCAACAGAAGCTCCCGCG-3′ | |
| -RW_R4 | (SEQ ID NO: 5) | |
| ATAD3A | 5′-ACACTCTTTCCCTACACGACGCTCT | |
| -RW- | TCCGATCTCCTGCAGCCACTCCCTGCT | |
| Illumina_F | C-3′ (SEQ ID NO: 6) | |
| ATAD3A | 5′-GACTGGAGTTCAGACGTGTGCTCTT | |
| -RW- | CCGATCTCCCTCAACAGAAGCTCCCGC | |
| Illumina_R | G-3′ (SEQ ID NO: 7) | |
| ATAD3B | 5′-CCGGCCACAGAAGGAAAACGGTG-3′ | ATAD3B |
| -RW_F1 | (SEQ ID NO: 8) | Exon15 |
| ATAD3B | 5′-CCCTCAACAGAAGCTCCCACA-3′ | |
| -RW_R3 | (SEQ ID NO: 9) | |
| ATAD3B-RW- | 5′-ACACTCTTTCCCTACACGACGCTCTT | |
| Illumina_F | CCGATCTCCGGCCACAGAAGGAAAACGGT | |
| | G-3′ (SEQ ID NO: 10) | |
| ATAD3B-R_W- | 5′-GACTGGAGTTCAGACGTGTGCTCTTC | |
| IlluminaR | CGATCTCCCTCAACAGAAGCTCCCACA-3′ | |
| | (SEQ ID NO: 11) | |
| ATAD3A | 5′-TTCCCGAGGAGCCGAGTCTG-3′ | ATAD3A |
| -TM_F2 | (SEQ ID NO: 12) | Exon10 |
| ATAD3A | 5-GCTCTGCCCAGCGTCCCTGC-3′ | |
| -TM_R1 | (SEQ ID NO: 13) | |
| ATAD3A-TM- | 5′-ACACTCTTTCCCTACACGACGCTCT | |
| Illumina_F | TCCGATCTTTCCCGAGGAGCCGAGTCT | |
| | G-3′ | |
| | (SEQ ID NO: 14) | |
| ATAD3A-TM- | 5′-GACTGGAGTTCAGACGTGTGCTCTTC | |
| Illumina_R | CGATCTGCTCTGCCCAGCGTCCCTGC-3′ | |
| | (SEQ ID NO: 15) | |
| ATAD3B- | 5′-CCCTGTCACCGAGGCTTCCG-3′ | ATAD3B |
| TM_F1 | (SEQ ID NO: 16) | Exon10 |
| ATAD3B- | 5′-AGAAGAGTGAGGGGAGACAGAA-3′ | |
| TM_R2 | (SEQ ID NO: 17) | |

TABLE 5
List of siRNAs and their corresponding
sequences

| siRNA | Duplex Sequences |
|---|---|
| Scrambled | 5′-CGUUAAUCGCGUAUAAUACGCGUAT-3′ |
| | (SEQ ID NO: 18) |
| | 3′-CAGCAAUUAGCGCAUAUUAUGCGCAUA-5′ |
| | (SEQ ID NO: 19) |
| siRAD51 | 5′-GUCACAAACUGAUCUAAAAUGUUTA-3′ |
| | (SEQ ID NO: 20) |
| | 3′-AUCAGUGUUUGACUAGAUUUUACAAAU-5′ |
| | (SEQ ID NO: 21) |
| siBRCA1 | 5′-GUACGAGAUUUAGUCAACUUGUUGA-3′ |
| | (SEQ ID NO: 22) |
| | 3′-UUCAUGCUCUAAAUCAGUUGAACAACU-5′ |
| | (SEQ ID NO: 23) |
| siBRCA2 | 5′-CAAGAAGCAUGUCAUGGUAAUACTT-3′ |
| | (SEQ ID NO: 24) |
| | 3′-GAGUUCUUCGUACAGUACCAUUAUGAA-5′ |
| | (SEQ ID NO: 25) |
| siCtIP | 5′-GAGAAUGUUUUAGAUGACAUAAAGA-3′ |
| | (SEQ ID NO: 26) |
| | 3′-GACUCUUACAAAAUCUACUGUAUUUCU-5′ |
| | (SEQ ID NO: 27) |

TABLE 6
List of primers used for qRT-PCR

| Primer for | |
|---|---|
| qRT-PCR | Sequences |
| RAD51-F | 5′-AGACCGAGCCCTAAGGAGAG-3′ |
| | (SEQ ID NO: 28) |
| RAD51-R | 5′-CTTCTCTACTCGCTTGCCCC-3′ |
| | (SEQ ID NO: 29) |
| CtIP-F | 5′-CAGAATAGGACTGAGTACGGTA |
| | AAG-3′ (SEQ ID NO: 30) |
| CtIP-R | 5′-CTGACTGCCATCCTTTGTATC |
| | T-3′ (SEQ ID NO: 31) |
| BRCA1-F | 5′-AAGCTGACAGATGGTTCATT-3′ |
| | (SEQ ID NO: 32) |
| BRCA1-R | 5′-ACAGGTTCCTTGATCAACTC-3′ |
| | (SEQ ID NO: 33) |
| BRCA2-F | 5′-TGTGGAAGTTGCGTATTGTA-3′ |
| | (SEQ ID NO: 34) |
| BRCA2-R | 5′-TAAATCTGATGATGGACGCC-3′ |
| | (SEQ ID NO: 35) |

Genomic PCR for Sanger Sequencing and Amplicon NGS. Genomic DNA was purified by using the PureLink™ Genomic DNA kit (Invitrogen, Cat #K182002) according to the manufacturer's protocol. For siRNA treated iPSCs, AllPrep DNA/RNA Micro Kit (Qiagen, Cat #80284) was used to purify both genomic DNA and RNA. For PCR amplification of the genomic region flanking ATAD3A exon 15 and exon 10, and ATAD3B exon 15 and exon 10, primers were designed via UCSC in silico PCR (genome.ucsc.edu/cgi-bin/hgPcr, Table 4). For Next Generation Sequencing, partial Illumina® adaptor sequences were added to the primers (Table 4). PCR reactions were performed using Q5® High-Fidelity DNA Polymerase (NEB Cat #M0491L). PCR products were purified with QIAquick PCR Purification kit (QIAGEN Cat #28106) or QIAquick Gel Extraction Kit (QIAGEN Cat #28706). Sanger sequencing of the PCR products was performed using Azenta Life Science (Burlington Massachusetts, USA). ICE (Inference of CRISPR Edits) analyses were used to determine HDR and indels frequencies. (Synthego Performance Analysis, ICE Analysis. 2019. v2.0. Synthego; [6.11.2021]). To analyze the contribution of gene-corrected sequences from Cas9-RNP treated iPSCs, the Sanger chromatogram of the same locus from SC20 was used as a control chromatogram, and c.1582C>T or c.1076C>T mock sequences were provided as donor templates on ICE analysis. Amplicon NGS was performed using the Amplicon-EZ service from Azenta Life Science.

Whole Genome Sequencing and Genomic Analysis. Genomic DNA was purified from iPSCs using PureLink Genomic DNA Mini Kit (ThermoFisher Cat #K182002) according to the manufacture's protocol. WGS library preparation was performed using TruSeq DNA PCR-free (550 bp). Sequencing was performed on NovaSeq6000 S4 150PE to target 30× mappable (100 Gb/sample). FASTQ was used to align files to the human reference genome GRCh37d5(ftp-trace.ncbi.nih.gov/1000genomes/ftp/technical/reference/phase2_reference_assembly_sequence/h s37d5.fa.gz) using BWA-MEM (version 0.7.12). Duplicate reads were removed using the mark duplicate command of PICARD (version 1.130). Indel realignment and base quality recalibration were performed using GATK3 (version 2015.1-3.4.0-1-ga5ca3fc), resulting in analysis-ready BAM files. IGV screenshots were taken using Integrative Genomics Viewer (version 2.8.2). Read depth Manhattan plots for copy number profile were generated using CNVpytor (70).

Sequence similarities between the ATAD3A target region and its equivalent regions in two paralogs, ATAD3B and ATAD3C, were obtained by Smith-Waterman pairwise sequence alignment using the EBI EMBOSS Water web server (www.cbi.ac.uk/Tools/psa/emboss_water). For input sequences, the inventors took the 320 bp upstream and downstream sequence (641 bps total length) proximal to the target site of ATAD3A and the equivalent ones of ATAD3B and ATAD3C.

Amplicon Sequencing Data Analysis. The amplicon sequencing paired reads were merged to single reads using PEAR (71). Subsequently, the merged reads were aligned to GRCh37d5 using BWA-MEM and sorted by samtools (version 1.9). All the reads that are misaligned with the left and right positions of the genomic region targeted by the primer pair were filtered out. Allelic information for the target sites were calculated using samtools mpileup with the minimum base quality set to 20 and the minimum mapping quality set to 20.

Generating Neural Progenitor Cells. Patient-derived iPSCs grown in mTeSR1 were pretreated with 10 μM Y-27632 for 30 minutes before dissociation. After dissociating with Accutase™, 3×10⁶ cells were plated onto each well of AggreWell™ 800 (StemCell Technologies Cat #34815) in STEMdiff™ Neural Induction Medium supplemented with SMADi (StemCell Technologies Cat #08581) and 10 μM Y-27632. Cells were cultured for five days at 37° C. in a 5% CO₂ humidified incubator to generate embryoid bodies (EB) with daily partial medium change. On day 5, EBs were plated on Matrigel-coated 6-well plates and cultured for seven days with daily full medium change to induce Neural Rosettes. On day 7, neural rosettes were selected by incubating with STEMdiff™ Neural Rosette Selection Reagent (StemCell Technologies Cat #05832) for 1.5 hours. Selected rosettes were re-plated on Matrigel-coated 6-well plates and cultured with daily full medium change until reaching ˜90% confluency. NPCs were grown in STEMdiff™ Neural Progenitor Medium after passaging to new wells of Matrigel-coated 6-well plates.

Measurement of Mitochondrial Function. The oxygen consumption rate (OCR) was measured using Seahorse XFe24 analyzer by following the manufacturer's protocol. Briefly, patient-derived and gene-corrected iPSCs were seeded at a density of 6×10⁴ cells per each well of Matrigel-coated XFe24 cell culture plates (Agilent 100777-004) a day before the measurement. The next day, cells were pre-incubated for an hour with complete XF DMEM medium (Agilent 103575-100) containing 10 mM glucose, 1 mM Sodium Pyruvate, and 2 mM L-Glutamine. Electron Transport Chain (ETC) inhibitors were used by following working concentration: 1 μM Oligomycin, 0.5 μM Carbonyl cyanide 4-(trifluoromethoxy) phenylhydrazone (FCCP), 1 μM Antimycin A. After the measurement, each well of cells was lysed with 10 μL RIPA buffer. The protein concentration measured with the Bradford assay. Raw data were normalized with protein concentration in the Agilent program and analyzed in Microsoft Excel.

Statistical Analysis. The GraphPad Prism and Excel software were used to process data, calculate statistics, and prepare graphs. The unpaired t-tests were used to determine statistical significance, with data presented as mean+SEM.

Flow cytometry. iPS cells grown in 6-well plates were harvested using 0.05% Trypsin-EDTA and stained for cells surface antigens using combinations of the following antibodies: IgG1 Alexa 488 negative control (AbD Serotec MCA2356A488), 1:10; anti-CD29 Alexa 488 (AbD Serotec MCA2298A488), 1:10; or IgG3 anti-SSEA4-APC (R&D FAB1435A), 1:10. Cells then fixed with 2% paraformaldehyde, and permeabilized with 0.1% Saponin and 0.1% BSA in DPBS. Nuclear antigens were stained with mouse IgG1-PE negative control (BD 559320), 1:10; or mouse IgG1 anti-OCT4-PE (BD 560186), 1:10. Stained cells were detected by cell cytometry (BD LSRII Analyzer) and the data was analyzed with BD FACS Diva software.

iPSCs Karyotyping. For C7, C10, C27, and C30 iPSC lines, karyotyping by G-banding was performed by Baylor Genetics (Houston, Texas, USA). For TM-C9 and TM-C21 iPSC lines, KaryoStat™ assay was performed by Thermo Fisher Scientific (Carlsbad, California, USA).

NPCs Immunostaining. 5×10⁵ NPCs were seeded on Matrigel-coated coverslip in 12-well plates and grown for 2 days at 37° C. in a 5% CO₂ humidified incubator. After washing twice with 1×phosphate-buffered saline (PBS), pH 7.4, cells were fixed for 10 minutes with 4% formaldehyde (Thermo Fisher Cat #F79500) in PBS. The primary antibodies were used overnight at the following dilutions: rabbit anti-Pax6 1:300 (BioLegend Cat #901301, RRID: AB_2565003), rabbit anti-Sox 1 1:300 (CellSignaling Technologies Cat #4194, RRID: AB_1904140), mouse anti-Nestin 1:1000 (Millipore Cat #MAB5326, RRID: AB_2251134). Secondary antibodies were used at 1:300: Alexa 488-conjugated anti-rabbit (Invitrogen Cat #A21206, RRID: AB_2535792), Alexa 568-conjugated anti-mouse (Invitrogen Cat #A11004, RRID: AB_2534072). Samples were mounted in Vectashield (Vector Labs Cat #H-1000, Burlingame, CA). Imaging was performed using the LSM880 confocal microscope (Zeiss). Images were processed with the Zeiss LSM Image Browser and Adobe Photoshop.

Quantitative real-time RT-PCR. Total RNA from iPSCs was extracted using the AllPrep® DNA/RNA Micro Kit (Qiagen, Cat #80284), followed by cDNA synthesis using iScript cDNA synthesis kit (Bio-Rad #1708891). Quantitative RT-PCR was performed using the FastStart Essential DNA Green Master (Roche #6402712001) and LightCycler® 96 Instrument (Roche). Amplification signals were normalized to GAPDH, and fold-changes were calculated using ΔΔCt method. Data analysis and calculations were performed using Excel (Microsoft). Primers used for qRT-PCR are in Table 6.

As embodied and broadly described herein, an aspect of the present disclosure relates to a method of introducing a genetic change in the genome of a cell with an exogenous DNA template-free Cas-based genome editing system comprising, consisting essentially of, or consisting of: identifying a target genomic sequence of the genome comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; introducing into the cell a variant-specific sgRNA that directs gene editing by gene conversion and nonallelic homologous recombination (NAHR) to cause a unidirectional transfer of genomic DNA from the homologous DNA sequences to the target genomic sequence, wherein the gene conversion or NAHR involves a double stranded break; and contacting the genome of the cell with the variant-specific sgRNA and a Cas-based genome editing system, thereby introducing the genetic conversion without the exogenous DNA template. In one aspect, the method of correcting a genetic error in the genome of a cell is in vivo or ex vivo. In another aspect, the genetic change restores the function of a gene. In another aspect, the genetic change corrects a disease-causing mutation. In another aspect, the pathogenic variants are selected from ATAD3A, ATAD3B, Survival of Motor Neuron 1 (SMN1), SMN2, CYP2D6/7, FCGR3A, HBB, HBD, HBG1/2, KRT86/81/83, KRT6B/C/A, HBA2/1, CLCNKA, CLCNKB, KRT14/16/17 or any genes with 88% or higher homology with one or more paralogs or pseudogenes. In another aspect, the target is not a region with microhomology or microduplication. In another aspect, a RNA-guided DNA endonuclease is selected from the group consisting of Cas9, Cas12a, Cas12b, Cas12c, Cas12d, Cas12c, Cas12h, Cas13a, Cas13b, Cas13c, Cpf1, and MAD7, or homologs, orthologs, or paralogs thereof. In another aspect, the sgRNA comprises a variant-specific sgRNA that targets a pathogenic variant immediately adjacent a protospacer adjacent motif (PAM) sequence. In another aspect, the Cas-based genome editing system comprises an RNA-guided DNA endonuclease selected from Streptococcus pyogenes Cas9 or a Staphylococcus aureus Cas9.

As embodied and broadly described herein, an aspect of the present disclosure relates to a method of introducing a genetic change in the genome of a cell with an exogenous DNA template-free system at a target site comprising, consisting essentially of, or consisting of: obtaining a nucleic acid comprising a variant-specific sgRNA specific for a target genomic sequence comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; introducing into the cell a variant-specific sgRNA that directs gene editing by gene conversion and nonallelic homologous recombination (NAHR) to cause a unidirectional transfer of genomic DNA from the homologous DNA sequences to the target genomic sequence, wherein the gene conversion or NAHR involves a double stranded break; and contacting the genome of the cell with the variant-specific sgRNA and a Cas-based genome editing system, thereby introducing the genetic conversion without an exogenous DNA template. In one aspect, the variant-specific sgRNA comprises the variant-specific sgRNA targets a pathogenic variant immediately adjacent to a protospacer adjacent motif (PAM) sequence. In another aspect, the Cas-based genome editing system comprises an RNA-guided DNA endonuclease selected from Streptococcus pyogenes Cas9 or a Staphylococcus aureus Cas9.

As embodied and broadly described herein, an aspect of the present disclosure relates to a method of making a variant-specific single guide RNA for exogenous DNA template-free gene editing comprising, consisting essentially of, or consisting of: (i) identifying a target genomic sequence of the genome comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; (ii) analyzing the nucleotide sequence and cut site with a computational model to identify a sequence for the variant-specific single guide RNA; (ii) synthesizing variant-specific single guide RNA, wherein the variant-specific sgRNA in the presence of Cas-based genome editing enzymes edits the target genomic sequence. In one aspect, the variant-specific sgRNA comprises one or more modifications. In another aspect, the modifications are selected from the group consisting of: nucleoside analogs, chemically modified bases, intercalated bases, modified sugars, and modified phosphate group linkers. In another aspect, the guide RNA further comprises one or more phosphorothioate, 5′-N-phosphporamidite linkages, or both.

As embodied and broadly described herein, an aspect of the present disclosure relates to a method of treating a genetic disease in a subject caused by a genetic error in the genome of one or more cells of the subject by introducing a genetic change in the genome of a cell with an exogenous DNA template-free genome editing system at a target site comprising, consisting essentially of, or consisting of: introducing into the cell a variant-specific sgRNA that directs gene editing by gene conversion and nonallelic homologous recombination (NAHR) to cause a unidirectional transfer of genomic DNA from the homologous DNA sequences to the target genomic sequence, wherein the gene conversion or NAHR involves a double-stranded break, wherein the variant specific sgRNA is specific for a target genomic sequence comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; and contacting the genome of the cell with the variant-specific sgRNA and a Cas-based genome editing system, thereby introducing the genetic conversion without an exogenous DNA template. In one aspect, the method of correcting the genetic error in the genome of a cell is in vivo or ex vivo. In another aspect, the genetic change restores the function of a gene. In another aspect, the genetic change corrects a disease-causing mutation. In another aspect, the pathogenic variants are selected from ATAD3A, ATAD3B, Survival of Motor Neuron 1 (SMN1), SMN2, CYP2D6/7, FCGR3A, HBB, HBD, HBG1/2, KRT86/81/83, KRT6B/C/A, HBA2/1, CLCNKA, CLCNKB, KRT14/16/17 or any genes with 88% or higher homology with one or more paralogs or pseudogenes. In another aspect, the target site is not a region with microhomology or microduplication. In another aspect, an RNA-guided DNA endonuclease is selected from the group consisting of Cas9, Cas12a, Cas12b, Cas12c, Cas12d, Cas12c, Cas12h, Cas13a, Cas13b, Cas13c, Cpf1, and MAD7, or homologs, orthologs, or paralogs thereof. In another aspect, the variant-specific sgRNA targets a pathogenic variant immediately adjacent to a protospacer adjacent motif (PAM) sequence. In another aspect, the Cas-based genome editing system comprises an RNA-guided DNA endonuclease selected from Streptococcus pyogenes Cas9 or a Staphylococcus aureus Cas9.

As embodied and broadly described herein, an aspect of the present disclosure relates to a single guide RNA identified by the method claimed herein. In one aspect, the guide RNA comprises one or more modifications. In another aspect, the modifications are selected from the group consisting of: nucleoside analogs, chemically modified bases, intercalated bases, modified sugars, and modified phosphate group linkers. In another aspect, the single guide RNA further comprises one or more phosphorothioate, 5′-N-phosphporamidite linkages, or both.

As embodied and broadly described herein, an aspect of the present disclosure relates to a vector comprising, consisting essentially of, or consisting of: a nucleotide sequence encoding one or more guide RNAs claimed herein. As embodied and broadly described herein, an aspect of the present disclosure relates to a host cell comprising, consisting essentially of, or consisting of: a vector encoding one or more guide RNAs claimed herein. As embodied and broadly described herein, an aspect of the present disclosure relates to a Cas-based genome editing system comprising a Cas protein complexed with at least one guide RNA identified by the method of the present invention. In another aspect, the method further comprises, consists essentially of, or consists of: an expression vector having at least one expressible nucleotide sequence encoding a Cas protein and at least one other expressible nucleotide sequence encoding a guide RNA, and wherein the single guide RNA is identified by the method claimed herein.

As embodied and broadly described herein, an aspect of the present disclosure relates to a method comprising, consisting essentially of, or consisting of: a computational model for selecting a single guide RNA sequence for use with a Cas-based genome editing system that introduces a genetic change in a genome by gene conversion and nonallelic homologous recombination (NAHR), the method comprising: using a processor to identify a polynucleotide sequence for a variant-specific sgRNA specific for a target genomic sequence comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; and synthesizing the variant-specific sgRNA. In one aspect, the computational model is a neural network model having one or more hidden layers. In another aspect, the computational model is a deep learning computational model. In another aspect, the computational model is trained with experimental data to predict a probability of distribution of indel lengths for any given nucleotide sequence and cut site. In another aspect, the computational model is trained with experimental data to predict a probability of distribution of genotype frequencies for any given nucleotide sequence and cut site. In another aspect, the computational model comprises one or more training modules for evaluating experimental data. In another aspect, the computational model predicts genomic repair outcomes for any given input nucleotide sequence and cut site. In another aspect, the method further comprises the step of identifying the available cut sites comprises identifying one or more protospacer adjacent motif (PAM) sequences. In another aspect, the computational model is at least one of: a deep learning computational model; a neural network model having one or more hidden layers; is trained with experimental data to predict the probability of distribution of indel lengths for any given nucleotide sequence and cut site; is trained with experimental data to predict the probability of distribution of genotype frequencies for any given nucleotide sequence and cut site; comprises one or more training modules for evaluating experimental data; or predicts genomic repair outcomes for any given input nucleotide sequence and cut site.

As embodied and broadly described herein, an aspect of the present disclosure relates to a method of introducing a genetic change in the genome of a cell with an exogenous DNA template-free Cas-based genome editing system comprising, consisting essentially of, or consisting of: (i) selecting a single guide RNA (sgRNA) for use with a Cas-based genome editing system capable of introducing a genetic change into a nucleotide sequence of a target genomic location; (ii) identifying a target genomic sequence of the genome comprising 88% or higher homology with one or more paralogs or pseudogenes; wherein the target genomic sequence has one or more nucleotides that differ(s) when compared to a homologous DNA sequence in the one or more paralogs or pseudogenes (allele-specificity); and wherein the homologous region in the one or more paralogs or pseudogenes has a desired nucleotide sequence for transfer to the target genomic sequence after a double strand break (DSB) by Cas-based genome editing; (iii) introducing into the cell the variant-specific sgRNA that directs gene editing by gene conversion and nonallelic homologous recombination (NAHR) to cause a unidirectional transfer of genomic DNA from the homologous DNA sequences to the target genomic sequence, wherein the gene conversion or NAHR involves a double-stranded break; and (iv) contacting the genome of the cell with the variant-specific sgRNA and a Cas-based genome editing system, thereby introducing the genetic conversion without an exogenous DNA template.

It is contemplated that any embodiment discussed in this specification can be implemented

with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), clement(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

J. He, C. C. Mao, A. Reyes, H. Sembongi, M. Di Re, C. Granycome, A. B. Clippingdale,
M. Fearnley, M. Harbour, A. J. Robinson, S. Reichelt, J. N. Spelbrink, J. E. Walker, I.
J. Holt, The AAA+ protein ATAD3 has displacement loop binding properties and is involved in mitochondrial nucleoid organization. J Cell Biol 176, 141-146 (2007).
B. Gilquin, E. Taillebourg, N. Cherradi, A. Hubstenberger, O. Gay, N. Merle, N. Assard,
M. O. Fauvarque, S. Tomohiro, O. Kuge, J. Baudier, The AAA+ ATPase ATAD3A controls mitochondrial dynamics at the interface of the inner and outer membranes. Mol Cell Biol 30, 1984-1996 (2010).
T. Harel, W. H. Yoon, C. Garone, S. Gu, Z. Coban-Akdemir, M. K. Eldomery, J. E. Posey, S. N. Jhangiani, J. A. Rosenfeld, M. T. Cho, S. Fox, M. Withers, S. M. Brooks, T. Chiang, L. Duraine, S. Erdin, B. Yuan, Y. Shao, E. Moussallem, C. Lamperti, M. A. Donati, J. D. Smith, H. M. Mclaughlin, C. M. Eng, M. Walkiewicz, F. Xia, T. Pippucci,
P. Magini, M. Seri, M. Zeviani, M. Hirano, J. V. Hunter, M. Srour, S. Zanigni, R. A. Lewis, D. M. Muzny, T. E. Lotze, E. Boerwinkle, G. Baylor-Hopkins Center for Mendelian, G. University of Washington Center for Mendelian, R. A. Gibbs, S. E. Hickey, B. H. Graham, Y. Yang, D. Buhas, D. M. Martin, L. Potocki, C. Graziano, H. J. Bellen, J. R. Lupski, Recurrent De Novo and Biallelic Variation of ATAD3A, Encoding a Mitochondrial Membrane Protein, Results in Distinct Neurological Syndromes. Am J Hum Genet 99, 831-845 (2016).
Z. Y. Yap, Y. H. Park, S. B. Wortmann, A. C. Gunning, S. Ezer, S. Lec, L. Duraine, E. Wilichowski, K. Wilson, J. A. Mayr, M. Wagner, H. Li, U. Kini, E. D. Black, K. G. Monaghan, J. R. Lupski, S. Ellard, D. S. Westphal, T. Harel, W. H. Yoon, Functional interpretation of ATAD3A variants in neuro-mitochondrial phenotypes. Genome Med 13, 55 (2021).
A. C. Gunning, K. Strucinska, M. Munoz Oreja, A. Parrish, R. Caswell, K. L. Stals, R. Durigon, K. Durlacher-Betzer, M. H. Cunningham, C. M. Grochowski, J. Baptista, C. Tysoe, E. Baple, N. Lahiri, T. Homfray, I. Scurr, C. Armstrong, J. Dean, U. Fernandez Pelayo, A. W. E. Jones, R. W. Taylor, V. K. Misra, W. H. Yoon, C. F. Wright, J. R. Lupski, A. Spinazzola, T. Harel, I. J. Holt, S. Ellard, Recurrent De Novo NAHR Reciprocal Duplications in the ATAD3 Gene Cluster Cause a Neurogenetic Trait with Perturbed Cholesterol and Mitochondrial Metabolism. Am J Hum Genet 106, 272-279 (2020).
R. Desai, A. E. Frazier, R. Durigon, H. Patel, A. W. Jones, I. Dalla Rosa, N. J. Lake, A.
G. Compton, H. S. Mountford, E. J. Tucker, A. L. R. Mitchell, D. Jackson, A. Sesay, M. Di Re, L. P. van den Heuvel, D. Burke, D. Francis, S. Lunke, G. McGillivray, S. Mandelstam, F. Mochel, B. Keren, C. Jardel, A. M. Turner, P. Ian Andrews, J. Smeitink,
J. N. Spelbrink, S. J. Heales, M. Kohda, A. Ohtake, K. Murayama, Y. Okazaki, A. Lombes, I. J. Holt, D. R. Thorburn, A. Spinazzola, ATAD3 gene cluster deletions cause cerebellar dysfunction associated with altered mitochondrial DNA and cholesterol metabolism. Brain 140, 1595-1610 (2017).
H. M. Cooper, Y. Yang, E. Ylikallio, R. Khairullin, R. Woldegebriel, K. L. Lin, L. Euro,
E. Palin, A. Wolf, R. Trokovic, P. Isohanni, S. Kaakkola, M. Auranen, T. Lonnqvist, S. Wanrooij, H. Tyynismaa, ATPase-deficient mitochondrial inner membrane protein ATAD3A disturbs mitochondrial dynamics in dominant hereditary spastic paraplegia. Hum Mol Genet 26, 1432-1443 (2017).
A. E. Frazier, A. G. Compton, Y. Kishita, D. H. Hock, A. E. Welch, S. S. C. Amarasekera, R. Rius, L. E. Formosa, A. Imai-Okazaki, D. Francis, M. Wang, N. J. Lake, S. Tregoning, J. S. Jabbari, A. Lucattini, K. R. Nitta, A. Ohtake, K. Murayama, D.
J. Amor, G. McGillivray, F. Y. Wong, M. S. van der Knaap, R. Jeroen Vermeulen, E. J. Wiltshire, J. M. Fletcher, B. Lewis, G. Baynam, C. Ellaway, S. Balasubramaniam, K. Bhattacharya, M. L. Freckmann, S. Arbuckle, M. Rodriguez, R. J. Taft, S. Sadedin, M. J. Cowley, A. E. Minoche, S. E. Calvo, V. K. Mootha, M. T. Ryan, Y. Okazaki, D. A. Stroud, C. Simons, J. Christodoulou, D. R. Thorburn, Fatal perinatal mitochondrial cardiac failure caused by recurrent de novo duplications in the ATAD3 locus. Med (N Y) 2, 49-73 (2021).
I. Hanes, H. J. McMillan, Y. Ito, K. D. Kernohan, J. Lazier, M. A. Lines, D. A. Dyment, A splice variant in ATAD3A expands the clinical and genetic spectrum of Harel-Yoon syndrome. Neurol Genet 6, e452 (2020).
S. Peralta, A. Gonzalez-Quintana, M. Ybarra, A. Delmiro, R. Perez-Perez, J. Docampo, J. Arenas, A. Blazquez, C. Ugalde, M. A. Martin, Novel ATAD3A recessive mutation associated to fatal cerebellar hypoplasia with multiorgan involvement and mitochondrial structural abnormalities. Mol Genet Metab 128, 452-462 (2019).
N. Dorison, P. Gaignard, A. Bayot, A. Gelot, P. H. Becker, S. Fourati, E. Lebigot, P. Charles, T. Wai, P. Therond, A. Slama, Mitochondrial dysfunction caused by novel ATAD3A mutations. Mol Genet Metab 131, 107-113 (2020).
J. A. Doudna, E. Charpentier, Genome editing. The new frontier of genome engineering with CRISPR-Cas9. Science 346, 1258096 (2014).
P. D. Hsu, D. A. Scott, J. A. Weinstein, F. A. Ran, S. Konermann, V. Agarwala, Y. Li, E.
J. Fine, X. Wu, O. Shalem, T. J. Cradick, L. A. Marraffini, G. Bao, F. Zhang, DNA targeting specificity of RNA-guided Cas9 nucleases. Nat Biotechnol 31, 827-832 (2013).
M. Jinek, K. Chylinski, I. Fonfara, M. Hauer, J. A. Doudna, E. Charpentier, A programmable dual-RNA-guided DNA endonuclease in adaptive bacterial immunity. Science 337, 816-821 (2012).
S. P. Jackson, J. Bartek, The DNA-damage response in human biology and disease.
Nature 461, 1071-1078 (2009).
A. Rabai, L. Reisser, B. Reina-San-Martin, K. Mamchaoui, B. S. Cowling, A. S. Nicot, J. Laporte, Allele-Specific CRISPR/Cas9 Correction of a Heterozygous DNM2 Mutation Rescues Centronuclear Myopathy Cell Phenotypes. Mol Ther Nucleic Acids 16, 246-256 (2019).
C. Smith, L. Abalde-Atristain, C. He, B. R. Brodsky, E. M. Braunstein, P. Chaudhari, Y.
Y. Jang, L. Cheng, Z. Ye, Efficient and allele-specific genome editing of disease loci in human iPSCs. Mol Ther 23, 570-577 (2015).
L. Duret, N. Galtier, Biased gene conversion and the evolution of mammalian genomic landscapes. Annu Rev Genomics Hum Genet 10, 285-311 (2009).
J. M. Chen, D. N. Cooper, N. Chuzhanova, C. Ferec, G. P. Patrinos, Gene conversion: mechanisms, evolution and human disease. Nat Rev Genet 8, 762-775 (2007).
P. Stankiewicz, J. R. Lupski, Genome architecture, rearrangements and genomic disorders. Trends Genet 18, 74-82 (2002).
J. R. Lupski, 2018 Victor A. McKusick Leadership Award: Molecular Mechanisms for Genomic and Chromosomal Rearrangements. Am J Hum Genet 104, 391-406 (2019).
S. Yanovsky-Dagan, A. Frumkin, J. R. Lupski, T. Harel, CRISPR/Cas9-induced gene conversion between ATAD3 paralogs. HGG Adv 3, 100092 (2022).
E. R. Burnight, M. Gupta, L. A. Wiley, K. R. Anfinson, A. Tran, R. Triboulet, J. M. Hoffmann, D. L. Klaahsen, J. L. Andorf, C. Jiao, E. H. Sohn, M. K. Adur, J. W. Ross, R.
F. Mullins, G. Q. Daley, T. M. Schlaeger, E. M. Stone, B. A. Tucker, Using CRISPR-Cas9 to Generate Gene-Corrected Autologous iPSCs for the Treatment of Inherited Retinal Degeneration. Mol Ther 25, 1999-2013 (2017).
S. G. Giannelli, M. Luoni, V. Castoldi, L. Massimino, T. Cabassi, D. Angeloni, G. C. Demontis, L. Leocani, M. Andreazzoli, V. Broccoli, Cas9/sgRNA selective targeting of the P23H Rhodopsin mutant allele for treating retinitis pigmentosa by intravitreal AAV9.PHP.B-based delivery. Hum Mol Genet 27, 761-779 (2018).
B. Gyorgy, C. Nist-Lund, B. Pan, Y. Asai, K. D. Karavitaki, B. P. Kleinstiver, S. P. Garcia, M. P. Zaborowski, P. Solanes, S. Spataro, B. L. Schneider, J. K. Joung, G. S. G. Geleoc, J. R. Holt, D. P. Corey, Allele-specific gene editing prevents deafness in a model of dominant progressive hearing loss. Nat Med 25, 1123-1130 (2019).
Y. Yamamoto, T. Makiyama, T. Harita, K. Sasaki, Y. Wuriyanghai, M. Hayano, S. Nishiuchi, H. Kohjitani, S. Hirose, J. Chen, F. Yokoi, T. Ishikawa, S. Ohno, K. Chonabayashi, H. Motomura, Y. Yoshida, M. Horie, N. Makita, T. Kimura, Allele- specific ablation rescues electrophysiological abnormalities in a human iPS cell model of long-QT syndrome with a CALM2 mutation. Hum Mol Genet 26, 1670-1677 (2017).
X. Liang, J. Potter, S. Kumar, Y. Zou, R. Quintanilla, M. Sridharan, J. Carte, W. Chen,
N. Roark, S. Ranganathan, N. Ravinder, J. D. Chesnut, Rapid and highly efficient mammalian cell engineering via Cas9 protein transfection. J Biotechnol 208, 44-53 (2015).
S. Kim, D. Kim, S. W. Cho, J. Kim, J. S. Kim, Highly efficient RNA-guided genome editing in human cells via delivery of purified Cas9 ribonucleoproteins. Genome Res 24, 1012-1019 (2014).
D. Conant, T. Hsiau, N. Rossi, J. Oki, T. Maures, K. Waite, J. Yang, S. Joshi, R. Kelso,
K. Holden, B. L. Enzmann, R. Stoner, Inference of CRISPR Edits from Sanger Trace Data. CRISPR J 5, 123-130 (2022).
G. Jin, C. Xu, X. Zhang, J. Long, A. H. Rezaeian, C. Liu, M. E. Furth, S. Kridel, B. Pasche, X. W. Bian, H. K. Lin, Atad3a suppresses Pink1-dependent mitophagy to maintain homeostasis of hematopoietic progenitor cells. Nat Immunol 19, 29-40 (2018).
G. Pharaoh, K. Sataranatarajan, K. Street, S. Hill, J. Gregston, B. Ahn, C. Kinter, M. Kinter, H. Van Remmen, Metabolic and Stress Response Changes Precede Disease Onset in the Spinal Cord of Mutant SOD1 ALS Mice. Front Neurosci 13, 487 (2019).
H. Koike-Yusa, Y. Li, E. P. Tan, C. Velasco-Herrera Mdel, K. Yusa, Genome-wide recessive genetic screening in mammalian cells with a lentiviral CRISPR-guide RNA library. Nat Biotechnol 32, 267-273 (2014).
E. P. Tan, Y. Li, C. Velasco-Herrera Mdel, K. Yusa, A. Bradley, Off-target assessment of CRISPR-Cas9 guiding RNAs in human iPS and mouse ES cells. Genesis 53, 225-236 (2015).
M. van Overbeek, D. Capurso, M. M. Carter, M. S. Thompson, E. Frias, C. Russ, J. S. Reece-Hoyes, C. Nye, S. Gradia, B. Vidal, J. Zheng, G. R. Hoffman, C. K. Fuller, A. P. May, DNA Repair Profiling Reveals Nonrandom Outcomes at Cas9-Mediated Breaks. Mol Cell 63, 633-646 (2016).
M. Kosicki, K. Tomberg, A. Bradley, Repair of double-strand breaks induced by CRISPR-Cas9 leads to large deletions and complex rearrangements. Nat Biotechnol 36, 765-771 (2018).
C. Markouli, E. Couvreu De Deckersberg, M. Regin, H. T. Nguyen, F. Zambelli, A. Keller, D. Dziedzicka, J. De Kock, L. Tilleman, F. Van Nieuwerburgh, L. Franceschini,
K. Sermon, M. Geens, C. Spits, Gain of 20q11.21 in Human Pluripotent Stem Cells Impairs TGF-beta-Dependent Neuroectodermal Commitment. Stem Cell Reports 13, 163- 176 (2019).
B. Elliott, C. Richardson, J. Winderbaum, J. A. Nickoloff, M. Jasin, Gene conversion tracts from double-strand break repair in mammalian cells. Mol Cell Biol 18, 93-101 (1998).
D. G. Taghian, J. A. Nickoloff, Chromosomal double-strand breaks induce gene conversion at high frequency in mammalian cells. Mol Cell Biol 17, 6386-6393 (1997).
R. Mayle, I. M. Campbell, C. R. Beck, Y. Yu, M. Wilson, C. A. Shaw, L. Bjergbaek, J.
R. Lupski, G. Ira, DNA REPAIR. Mus81 and converging forks limit the mutagenicity of replication fork breakage. Science 349, 742-747 (2015).
N. Saini, S. Ramakrishnan, R. Elango, S. Ayyar, Y. Zhang, A. Deem, G. Ira, J. E. Haber,
K. S. Lobachev, A. Malkova, Migrating bubble during break-induced replication drives conservative DNA synthesis. Nature 502, 389-392 (2013).
C. C. Homem, M. Repic, J. A. Knoblich, Proliferation control in neural stem and progenitor cells. Nat Rev Neurosci 16, 647-659 (2015).
F. W. Alt, B. Schwer, DNA double-strand breaks as drivers of neural genomic change, function, and disease. DNA Repair (Amst) 71, 158-163 (2018).
M. Wang, P. C. Wei, C. K. Lim, I. S. Gallina, S. Marshall, M. C. Marchetto, F. W. Alt, F.
H. Gage, Increased Neural Progenitor Proliferation in a hiPSC Model of Autism Induces Replication Stress-Associated Genome Instability. Cell Stem Cell 26, 221-233 e226 (2020).
P. Kumar, S. Henikoff, P. C. Ng, Predicting the effects of coding non-synonymous variants on protein function using the SIFT algorithm. Nat Protoc 4, 1073-1081 (2009).
R. Prakash, Y. Zhang, W. Feng, M. Jasin, Homologous recombination and human health: the roles of BRCA1, BRCA2, and associated proteins. Cold Spring Harb Perspect Biol 7, a016600 (2015).
Y. Bai, L. S. Symington, A Rad52 homolog is required for RAD51-independent mitotic recombination in Saccharomyces cerevisiae. Genes Dev 10, 2025-2037 (1996).
D. N. Gallagher, N. Pham, A. M. Tsai, N. V. Janto, J. Choi, G. Ira, J. E. Haber, A Rad51-independent pathway promotes single-strand template repair in gene editing. PLOS Genet 16, €1008689 (2020).
G. Ira, J. E. Haber, Characterization of RAD51-independent break-induced replication that acts preferentially with short homologous sequences. Mol Cell Biol 22, 6384-6392 (2002).
R. L. Dilley, P. Verma, N. W. Cho, H. D. Winters, A. R. Wondisford, R. A. Greenberg, Break-induced telomere synthesis underlies alternative telomere maintenance. Nature 539, 54-58 (2016).
C. D. Richardson, K. R. Kazane, S. J. Feng, E. Zelin, N. L. Bray, A. J. Schafer, S. N. Floor, J. E. Corn, CRISPR-Cas9 genome editing in human cells occurs via the Fanconi anemia pathway. Nat Genet 50, 1132-1139 (2018).
A. K. Wong, P. A. Ormonde, R. Pero, Y. Chen, L. Lian, G. Salada, S. Berry, Q. Lawrence, P. Dayananth, P. Ha, S. V. Tavtigian, D. H. Teng, P. L. Bartel, Characterization of a carboxy-terminal BRCA1 interacting protein. Oncogene 17, 2279-2285 (1998).
X. Yu, L. C. Wu, A. M. Bowcock, A. Aronheim, R. Baer, The C-terminal (BRCT) domains of BRCA1 interact in vivo with CtIP, a protein implicated in the CtBP pathway of transcriptional repression. J Biol Chem 273, 25388-25392 (1998).
S. M. Sy, M. S. Huen, J. Chen, PALB2 is an integral component of the BRCA complex required for homologous recombination repair. Proc Natl Acad Sci USA 106, 7155-7160 (2009).
B. Xia, J. C. Dorsman, N. Ameziane, Y. de Vries, M. A. Rooimans, Q. Sheng, G. Pals, A. Errami, E. Gluckman, J. Llera, W. Wang, D. M. Livingston, H. Joenje, J. P. de Winter, Fanconi anemia is associated with a defect in the BRCA2 partner PALB2. Nat Genet 39, 159-161 (2007).
D. Egli, M. V. Zuccaro, M. Kosicki, G. M. Church, A. Bradley, M. Jasin, Inter-homologue repair in fertilized human eggs? Nature 560, E5-E7 (2018).
H. Y. Shin, C. Wang, H. K. Lee, K. H. Yoo, X. Zeng, T. Kuhns, C. M. Yang, T. Mohr, C. Liu, L. Hennighausen, CRISPR/Cas9 targeting events cause complex deletions and insertions at 17 sites in the mouse genome. Nat Commun 8, 15464 (2017).
G. Cullot, J. Boutin, J. Toutain, F. Prat, P. Pennamen, C. Rooryck, M. Teichmann, E. Rousseau, I. Lamrissi-Garcia, V. Guyonnet-Duperat, A. Bibeyran, M. Lalanne, V. Prouzet-Mauleon, B. Turcq, C. Ged, J. M. Blouin, E. Richard, S. Dabernat, F. Moreau-Gaudry, A. Bedel, CRISPR-Cas9 genome editing induces megabase-scale chromosomal truncations. Nat Commun 10, 1136 (2019).
D. R. Simeonov, A. J. Brandt, A. Y. Chan, J. T. Cortez, Z. Li, J. M. Woo, Y. Lee, C. M.
B. Carvalho, A. C. Indart, T. L. Roth, J. Zou, A. P. May, J. R. Lupski, M. S. Anderson, F.
W. Buaas, D. S. Rokhsar, A. Marson, A large CRISPR-induced bystander mutation causes immune dysregulation. Commun Biol 2, 70 (2019).
I. Hoijer, A. Emmanouilidou, R. Ostlund, R. van Schendel, S. Bozorgpana, M. Tijsterman, L. Feuk, U. Gyllensten, M. den Hoed, A. Ameur, CRISPR-Cas9 induces large structural variants at on-target and off-target sites in vivo that segregate across generations. Nat Commun 13, 627 (2022).
M. R. Lieber, The mechanism of double-strand DNA break repair by the nonhomologous DNA end-joining pathway. Annu Rev Biochem 79, 181-211 (2010).
J. Buis, T. Stoneham, E. Spehalski, D. O. Ferguson, Mrell regulates CtIP-dependent double-strand break repair by interaction with CDK2. Nat Struct Mol Biol 19, 246-252 (2012).
V. T. Chu, T. Weber, B. Wefers, W. Wurst, S. Sander, K. Rajewsky, R. Kuhn, Increasing the efficiency of homology-directed repair for CRISPR-Cas9-induced precise gene editing in mammalian cells. Nat Biotechnol 33, 543-548 (2015).
C. Yu, Y. Liu, T. Ma, K. Liu, S. Xu, Y. Zhang, H. Liu, M. La Russa, M. Xie, S. Ding, L.
S. Qi, Small molecules enhance CRISPR genome editing in pluripotent stem cells. Cell Stem Cell 16, 142-147 (2015).
B. S. Paulsen, P. K. Mandal, R. L. Frock, B. Boyraz, R. Yadav, S. Upadhyayula, P. Gutierrez-Martinez, W. Ebina, A. Fasth, T. Kirchhausen, M. E. Talkowski, S. Agarwal,
F. W. Alt, D. J. Rossi, Ectopic expression of RAD52 and dn53BP1 improves homology-directed repair during CRISPR-Cas9 genome editing. Nat Biomed Eng 1, 878-888 (2017).
M. Charpentier, A. H. Y. Khedher, S. Menoret, A. Brion, K. Lamribet, E. Dardillac, C. Boix, L. Perrouault, L. Tesson, S. Geny, A. De Cian, J. M. Itier, I. Anegon, B. Lopez, C. Giovannangeli, J. P. Concordet, CtIP fusion to Cas9 enhances transgene integration by homology-dependent repair. Nat Commun 9, 1133 (2018).
C. M. Carvalho, J. R. Lupski, Mechanisms underlying structural variant formation in genomic disorders. Nat Rev Genet 17, 224-238 (2016).
R. M. Liskay, A. Letsou, J. L. Stachelek, Homology requirement for efficient gene conversion between duplicated chromosomal sequences in mammalian cells. Genetics 115, 161-167 (1987).
A. S. Waldman, R. M. Liskay, Dependence of intrachromosomal recombination in mammalian cells on uninterrupted homology. Mol Cell Biol 8, 5350-5357 (1988).
L. T. Reiter, P. J. Hastings, E. Nelis, P. De Jonghe, C. Van Broeckhoven, J. R. Lupski, Human meiotic recombination products revealed by sequencing a hotspot for homologous strand exchange in multiple HNPP deletion patients. Am J Hum Genet 62, 1023-1033 (1998).
M. Suvakov, A. Panda, C. Diesh, I. Holmes, A. Abyzov, CNVpytor: a tool for copy number variation detection and analysis from read depth and allele imbalance in whole-genome sequencing. Gigascience 10, (2021).
J. Zhang, K. Kobert, T. Flouri, A. Stamatakis, PEAR: a fast and accurate Illumina Paired-End reAd mergeR. Bioinformatics 30, 614-620 (2014).

What is claimed is:

1. A method of introducing a genetic change in a genome of a cell with an exogenous DNA template-free Cas-based genome editing system comprising:
identifying a target genomic sequence of the genome comprising 88% or higher homology with one or more paralogs or pseudogenes;
wherein the target genomic sequence has one or more nucleotides that differ(s) when compared to a homologous DNA sequence in the one or more paralogs or pseudogenes (allele-specificity); and
wherein a homologous region in the one or more paralogs or pseudogenes has a desired nucleotide sequence for transfer to the target genomic sequence after a double strand break (DSB) by Cas-based genome editing;
introducing into the cell a variant-specific sgRNA that directs gene editing by gene conversion and nonallelic homologous recombination (NAHR) to cause a unidirectional transfer of genomic DNA from the homologous DNA sequences to the target genomic sequence, wherein a gene conversion or NAHR involves a double stranded break; and
contacting the genome of the cell with the variant-specific sgRNA and a Cas-based genome editing system, thereby introducing a genetic conversion without the exogenous DNA template.

2. The method of claim 1, wherein the method of correcting a genetic error in the genome of a cell is in vivo or ex vivo.

3. The method of claim 1, wherein the genetic change restores gene function.

4. The method of claim 1, wherein the genetic change corrects a disease-causing mutation.

5. The method of claim 1, wherein pathogenic variants are selected from ATAD3A, ATAD3B, Survival of Motor Neuron 1 (SMN1), SMN2, CYP2D6/7, FCGR3A, HBB, HBD, HBG1/2, KRT86/81/83, KRT6B/C/A, HBA2/1, CLCNKA, CLCNKB, KRT14/16/17 or any genes with 88% or higher homology with one or more paralogs or pseudogenes.

6. The method of claim 1, wherein the target is not a region with microhomology or microduplication.

7. The method of claim 1, wherein a RNA-guided DNA endonuclease is selected from the group consisting of Cas9, Cas12a, Cas12b, Cas12c, Cas12d, Cas12e, Cas12h, Cas13a, Cas13b, Cas13c, Cpf1, and MAD7, or homologs, orthologs, or paralogs thereof.

8. The method of claim 1, wherein the sgRNA comprises a variant-specific sgRNA that targets a pathogenic variant immediately adjacent a protospacer adjacent motif (PAM) sequence.

9. The method of claim 1, wherein the Cas-based genome editing system comprises an RNA-guided DNA endonuclease selected from Streptococcus pyogenes Cas9 or a Staphylococcus aureus Cas9.

10. A method of introducing a genetic change in a genome of a cell with an exogenous DNA template-free system at a target site comprising:
obtaining a nucleic acid comprising a variant-specific sgRNA specific for a target genomic sequence comprising 88% or higher homology with one or more paralogs or pseudogenes;
wherein the target genomic sequence has one or more nucleotides that differ(s) when compared to a homologous DNA sequence in the one or more paralogs or pseudogenes (allele-specificity); and
wherein a homologous region in the one or more paralogs or pseudogenes has a desired nucleotide sequence for transfer to the target genomic sequence after a double strand break (DSB) by Cas-based genome editing;
introducing into the cell a variant-specific sgRNA that directs gene editing by gene conversion and nonallelic homologous recombination (NAHR) to cause a unidirectional transfer of genomic DNA from the homologous DNA sequences to the target genomic sequence, wherein gene conversion or NAHR involves a double stranded break; and
contacting the genome of the cell with the variant-specific sgRNA and a Cas-based genome editing system, thereby introducing a genetic conversion without an exogenous DNA template.

11. The method of claim 10, wherein the variant-specific sgRNA comprises the variant-specific sgRNA targets a pathogenic variant immediately adjacent to a protospacer adjacent motif (PAM) sequence.

12. The method of claim 10, wherein the Cas-based genome editing system comprises an RNA-guided DNA endonuclease selected from Streptococcus pyogenes Cas9 or a Staphylococcus aureus Cas9.

13. A method of making a variant-specific single guide RNA for exogenous DNA template-free gene editing comprising:
(i) identifying a target genomic sequence of the genome comprising 88% or higher homology with one or more paralogs or pseudogenes;
wherein the target genomic sequence has one or more nucleotides that differ(s) when compared to a homologous DNA sequence in the one or more paralogs or pseudogenes (allele-specificity); and
wherein a homologous region in the one or more paralogs or pseudogenes has a desired nucleotide sequence for transfer to the target genomic sequence after a double strand break (DSB) by Cas-based genome editing;
(ii) analyzing the nucleotide sequence and cut site with a computational model to identify a sequence for the variant-specific single guide RNA;
(ii) synthesizing variant-specific single guide RNA, wherein the variant-specific sgRNA in a presence of Cas-based genome editing enzymes edits the target genomic sequence.

14. The method of claim 13, wherein the variant-specific sgRNA comprises one or more modifications.

15. The method of claim 14, wherein the modifications are selected from the group consisting of: nucleoside analogs, chemically modified bases, intercalated bases, modified sugars, and modified phosphate group linkers.

16. The method of claim 13, wherein the guide RNA further comprises one or more phosphorothioate, 5′-N-phosphporamidite linkages, or both.

17. A method of treating a genetic disease in a subject caused by a genetic error in a genome of one or more cells of the subject by introducing a genetic change in the genome of a cell with an exogenous DNA template-free genome editing system at a target site comprising:
introducing into the cell a variant-specific sgRNA that directs gene editing by gene conversion and nonallelic homologous recombination (NAHR) to cause a unidirectional transfer of genomic DNA from a homologous DNA sequences to a target genomic sequence, wherein the gene conversion or NAHR involves a double-stranded break,
wherein the variant specific sgRNA is specific for a target genomic sequence comprising 88% or higher homology with one or more paralogs or pseudogenes;
wherein the target genomic sequence has one or more nucleotides that differ(s) when compared to a homologous DNA sequence in the one or more paralogs or pseudogenes (allele-specificity); and
wherein a homologous region in the one or more paralogs or pseudogenes has a desired nucleotide sequence for transfer to the target genomic sequence after a double strand break (DSB) by Cas-based genome editing; and
contacting the genome of the cell with the variant-specific sgRNA and a Cas-based genome editing system, thereby introducing a genetic conversion without an exogenous DNA template.

18. The method of claim 17, wherein the method of correcting the genetic error in the genome of a cell is in vivo or ex vivo.

19. The method of claim 17, wherein the genetic change restores gene function.

20. The method of claim 17, wherein the genetic change corrects a disease-causing mutation.

21. The method of claim 17, wherein pathogenic variants are selected from ATAD3A, ATAD3B, Survival of Motor Neuron 1 (SMN1), SMN2, CYP2D6/7, FCGR3A, HBB, HBD, HBG1/2, KRT86/81/83, KRT6B/C/A, HBA2/1, CLCNKA, CLCNKB, KRT14/16/17 or any genes with 88% or higher homology with one or more paralogs or pseudogenes.

22. The method of claim 17, wherein the target site is not a region with microhomology or microduplication.

23. The method of claim 17, wherein an RNA-guided DNA endonuclease is selected from the group consisting of Cas9, Cas12a, Cas12b, Cas12c, Cas12d, Cas12e, Cas12h, Cas13a, Cas13b, Cas13c, Cpf1, and MAD7, or homologs, orthologs, or paralogs thereof.

24. The method of claim 17, wherein the variant-specific sgRNA targets a pathogenic variant immediately adjacent to a protospacer adjacent motif (PAM) sequence.

25. The method of claim 17, wherein the Cas-based genome editing system comprises an RNA-guided DNA endonuclease selected from Streptococcus pyogenes Cas9 or a Staphylococcus aureus Cas9.

26. A single guide RNA identified by the method of claim 1.

27. The single guide RNA of claim 26, wherein the guide RNA comprises one or more modifications.

28. The single guide RNA of claim 27, wherein the modifications are selected from the group consisting of: nucleoside analogs, chemically modified bases, intercalated bases, modified sugars, and modified phosphate group linkers.

29. The single guide RNA of claim 26, wherein the single guide RNA further comprises one or more phosphorothioate, 5′-N-phosphporamidite linkages, or both.

30. A vector comprising a nucleotide sequence encoding one or more guide RNAs of claim 26.

31. A host cell comprising a vector encoding one or more guide RNAs of claim 26.

32. A Cas-based genome editing system comprising a Cas protein complexed with at least one guide RNA identified by the method of claim 1.

33. The Cas-based genome editing system of claim 32, comprising an expression vector having at least one expressible nucleotide sequence encoding a Cas protein and at least one other expressible nucleotide sequence encoding a guide RNA, and wherein a single guide RNA is designed by:
identifying a target genomic sequence of the genome comprising 88% or higher homology with one or more paralogs or pseudogenes;
wherein the target genomic sequence has one or more nucleotides that differ(s) when compared to a homologous DNA sequence in the one or more paralogs or pseudogenes (allele-specificity); and
wherein a homologous region in the one or more paralogs or pseudogenes has a desired nucleotide sequence for transfer to the target genomic sequence after a double strand break (DSB) by Cas-based genome editing;
introducing into the cell a variant-specific sgRNA that directs gene editing by gene conversion and nonallelic homologous recombination (NAHR) to cause a unidirectional transfer of genomic DNA from the homologous DNA sequences to the target genomic sequence, wherein a gene conversion or NAHR involves a double stranded break.

34. A method comprising a computational model for selecting a single guide RNA sequence for use with a Cas-based genome editing system that introduces a genetic change in a genome by gene conversion and nonallelic homologous recombination (NAHR), the method comprising:
using a processor to identify a polynucleotide sequence for a variant-specific sgRNA specific for a target genomic sequence comprising 88% or higher homology with one or more paralogs or pseudogenes;
wherein the target genomic sequence has one or more nucleotides that differ(s) when compared to a homologous DNA sequence in the one or more paralogs or pseudogenes (allele-specificity); and
wherein a homologous region in the one or more paralogs or pseudogenes has a desired nucleotide sequence for transfer to the target genomic sequence after a double strand break (DSB) by Cas-based genome editing; and
synthesizing the variant-specific sgRNA.

35. The method of claim 34, wherein the computational model is a neural network model having one or more hidden layers.

36. The method of claim 34, wherein the computational model is a deep learning computational model.

37. The method of claim 34, wherein the computational model is trained with experimental data to predict a probability of distribution of indel lengths for any given nucleotide sequence and cut site.

38. The method of claim 34, wherein the computational model is trained with experimental data to predict a probability of distribution of genotype frequencies for any given nucleotide sequence and cut site.

39. The method of claim 34, wherein the computational model comprises one or more training modules for evaluating experimental data.

40. The method of claim 34, wherein the computational model predicts genomic repair outcomes for any given input nucleotide sequence and cut site.

41. The method of claim 34, further comprising the step of identifying one or more available cut sites comprises identifying one or more protospacer adjacent motif (PAM) sequences.

42. The method of claim 34, wherein the computational model is at least one of:
a deep learning computational model;
a neural network model having one or more hidden layers;
is trained with experimental data to predict a probability of distribution of indel lengths for any given nucleotide sequence and cut site;
is trained with experimental data to predict the probability of distribution of genotype frequencies for any given nucleotide sequence and cut site;
comprises one or more training modules for evaluating experimental data; or
predicts genomic repair outcomes for any given input nucleotide sequence and cut site.

43. A method of introducing a genetic change in a genome of a cell with an exogenous DNA template-free Cas-based genome editing system comprising:
(i) selecting a single guide RNA (sgRNA) for use with a Cas-based genome editing system capable of introducing a genetic change into a nucleotide sequence of a target genomic location;
(ii) identifying a target genomic sequence of the genome comprising 88% or higher homology with one or more paralogs or pseudogenes;
wherein the target genomic sequence has one or more nucleotides that differ(s) when compared to a homologous DNA sequence in the one or more paralogs or pseudogenes (allele-specificity); and
wherein a homologous region in the one or more paralogs or pseudogenes has a desired nucleotide sequence for transfer to the target genomic sequence after a double strand break (DSB) by Cas-based genome editing;
(iii) introducing into the cell a variant-specific sgRNA that directs gene editing by gene conversion and nonallelic homologous recombination (NAHR) to cause a unidirectional transfer of genomic DNA from the homologous DNA sequences to the target genomic sequence, wherein the gene conversion or NAHR involves a double-stranded break; and
(iv) contacting the genome of the cell with the variant-specific sgRNA and a Cas-based genome editing system, thereby introducing a genetic conversion without an exogenous DNA template.
